(19) United States
(12) Patent Application Publication
(10) Pub. No.: US 2004/0260071 A1
(43) Pub. Date: Dec. 23, 2004

(54) Benzophene-linked crf and crf-like peptides for covalent labeling of corticotropin-releasing factor crf binding protein

(76) Inventors: Klaus Eckart, Friedland (DE); Joachim Spiess, Gottingen (DE); Olaf Jahn, Cottingen (DE)

Correspondence Address: BIRCH STEWART KOLASCH & BIRCH, PO BOX 747, FALLS CHURCH, VA, 22040-0747, US

(21) Appl. No.: 10/479,705

(22) PCT Filed: May 27, 2002
(86) PCT No.: PCT/EP02/05798

(30) Foreign Application Priority Data
May 25, 2001 (EP) 01112745.3

(51) Int. Cl. C07K 14/475

(52) U.S. Cl. 530/399

(57) ABSTRACT

The present invention relates to a ligand of the Corticotropin-Releasing Factor (CRF)-binding protein (CRFBP) selected from the group consisting of CRF, Urocortin (Ucn), and Urotensin, said ligand comprising a covalently linked benzophenone moiety. Further, the present invention relates to a process for the purification of a CRFBP which comprises reacting said CRFBP with the ligand of the invention, performing photoaffinity labeling, and purifying the resultant photoreaction products by HPLC. The present invention also relates to the use of the ligand of the invention for detecting CRFBP and for identifying the binding site within CRFBP. The present invention also relates to a method for identifying an inhibitor of the binding of ARF to CRFBP.

The present invention relates to a ligand of the Corticotropin-Releasing Factor (CRF)-binding protein (CRFBP) selected from the group consisting of CRF, Urocortin (Ucn), and Urotensin, said ligand comprising a covalently linked benzophenone moiety. Further, the present invention relates to a process for the purification of a CRFBP which comprises reacting said CRFBP with the ligand of the invention, performing photoaffinity labeling, and purifying the resultant photoreaction products by HPLC. The present invention also relates to the use of the ligand of the invention for detecting CRFBP and for identifying the binding site within CRFBP. The present invention also relates to a method for identifying an inhibitor of the binding of CRF to CRFBP.

Several documents are cited throughout the text of this specification either by name or are referred to by numerals to within parenthesis. Each of the documents cited herein (including any manufacturer's specifications, instructions, etc.) are hereby incorporated herein by reference; however, there is no admission that any document cited is indeed prior art as to the present invention.

Corticotropin-releasing factor (CRF) is known as the key mediator of the endocrine, autonomic, immunologic, and behavioral responses to stress. The 41 amino acid residue C-terminally amidated peptide (1) was originally identified on the basis of its ability to stimulate the secretion of adrenocorticotropic hormone (ACTH) from the anterior pituitary (2). Secreted ACTH then triggers the release of glucocorticoids from the adrenal cortex. Thus, the release of glucocorticoids after exposure to stress is integrated by the hypothalamus-pituitary-adrenal (HPA) axis with CRF as the main modulator.

CRF as well as the CRF-like peptide urocortin (Ucn) (3) act through two distinct G protein-coupled receptors, CRF receptor 1 and 2 (CRFR1, CRFR2; for review see: (4)). In addition, a soluble structurally unrelated 37 kD CRF binding protein (CRFBP) (5, 6) is able to bind human/rat CRF (h/rCRF) and Ucn but not ovine CRF (oCRF) with high affinity. In primates, CRFBP mRNA is found in the brain, pituitary, liver and placenta, whereas in rodents it is exclusively detected in the brain and pituitary (6, 7). Present evidence indicates that CRFBP may act through a trapping mechanism for CRF and Ucn and thereby functions as a negative regulator of the synaptic or endocrine actions of these peptides (8).

In the CNS, CRF is involved in the modulation of locomotor activity, food intake, learning and anxiety and is assumed to be also associated with a number of neuropsychiatric diseases (9). Recent therapeutical interest in the CRFBP is related to certain synthetic fragments of h/rCRF to function as ligand inhibitors. These compounds such as h/rCRF⁶⁻³³ (10), which are inactive at the CRF receptors, are capable to displace endogenous CRF from CRFBP. Consequently, central administration of h/rCRF⁶⁻³³ selectively enhances by release of endogenous CRF the learning abilities of rats without producing anxiety or appetite suppression and is therefore proposed as a possible treatment of cognitive deficits such as Alzheimer's disease (11). However, peptides such as h/rCRF⁶⁻³³ may not be able to pass the blood-brain barrier. Furthermore, their half-life time in biological fluids is limited due to the fact that no modifications creating resistance against endo- and exopeptidase degradation were introduced.

A detailed knowledge of the interaction of CRF-like peptides and CRFBP on the structural level would, therefore, be helpful for the development of new peptidic and non-peptidic CRFBP ligand inhibitors.

Thus, the technical problem underlying the present invention was to provide means and methods for the identification of new peptidic and/or non-peptidic CRFBP ligand inhibitors.

The solution to said technical problem is provided by the embodiments characterized in the claims.

It is therefore an object of the present invention to provide photoactivatable CRF analogs containing benzophenone photophores in different positions which can be used to identify ligand binding sites of CRFBP by photoaffinity-labeling followed by mass spectrometric characterization of the photoadducts. Thereby, photoaffinity labeling (PAL) combined with mass spectrometric characterization of the photoadduct may be used to identify contact sites between CRFBP and its ligands.

The benzophenone photophore was chosen as the most promising photoactivatable group, because in contrast to aryldiazirines, it reacts even in the presence of solvent water preferentially with unreactive C—H bonds (13). This is of importance in order to obtain a stable covalent linkage that survives the applied analytical strategy. Thus, the new photoactivatable CRF analogs are compared with the aryldiazirine photoprobe 4-(1-azi-2,2,2-trifluoroethyl)benzoyl-[¹²⁵I]-Tyr⁰-h/rCRF (Diaz-Tyr⁰-h/rCRF) previously used (14).

Photoaffinity Labeling (PAL)

1 nM CRFBP was incubated with a 100 fold molar excess of photoactivatable ligand for 1 hour at room temperature. This CRFBP concentration was similar to the level of CRFBP detected in human plasma. Similar concentrations of about 0.1 to 0.2 nM CRFBP were also found to be best for the CRFBP binding assay system. The irradiation was carried out with UV-light of wavelenghts greater than 300 nm in an ice bath. Thereby, photodestruction of the protein was prevented and heating of the sample was minimized. The highest photoadduct yields of about 65% were found after 90 minutes irradiation time. Higher concentrations of CRFBP resulted in significantly decreased photoadduct yields.

The establishment of the newly developed PAL procedure including the characterization of the photoadduct is also exemplarily demonstrated in the appended examples.

Affinity of the Photoactivatable CRF Analogs to rCRFBP

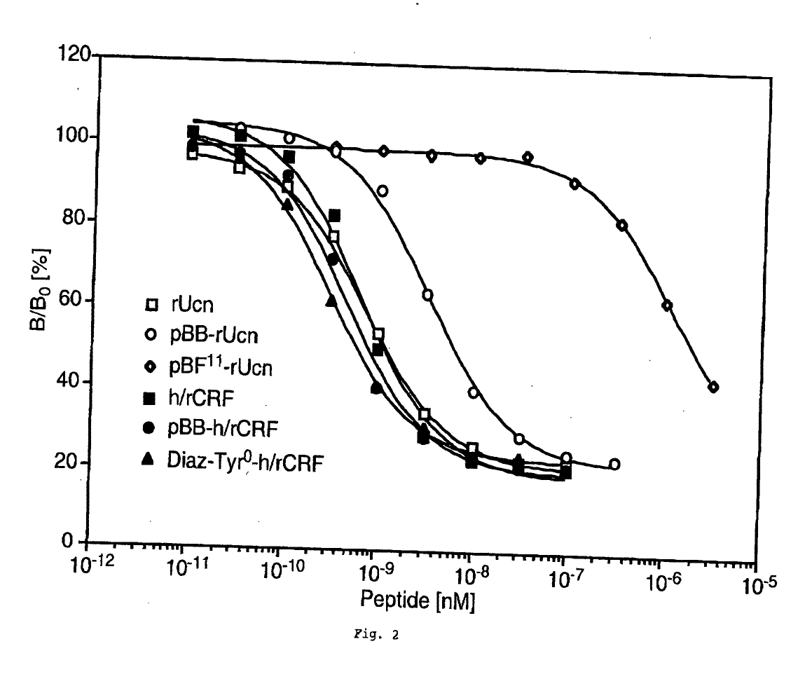

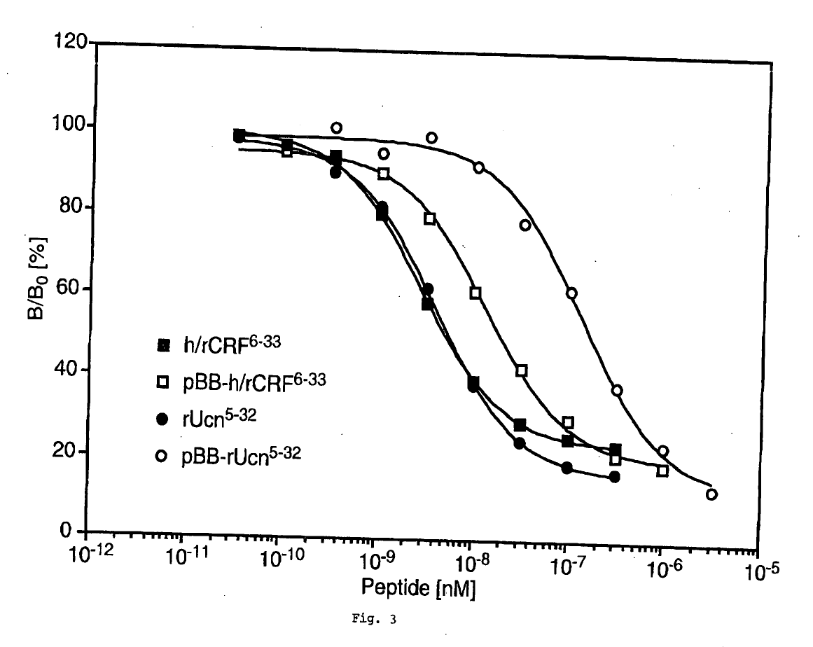

A prerequisite for experiments using the PAL technique is that a photoactivatable ligand retains its high affinity to the receptor protein to be labeled. Therefore, the inhibition constants (K_{I}) (Table 1a) or the IC₅₀ values (Table 1b) of some of the photoactivatable CRF analogs were determined in comparison to the unmodified peptides (see also FIG. 2, FIG. 3). On the basis of homologous competition experiments, a K_{D} of 0.17±0.003 nM (mean±S.E.M., n=3) was calculated for rUcn at rCRFBP which is in good agreement with the value found for recombinant human CRFBP (21). Comparison of the K_{I} values of rUcn, h/rCRF and their respective derivatives (Table 1a, compound 1-5; FIG. 2) revealed that N-terminal modifications with a photophore were tolerated in all full length-peptides. Consequently, pBB-rUcn, pBB-h/rCRF, and Diaz-Tyr⁰-h/rCRF could be considered as high affinity ligands (K_{I}<1 nM). In contrast, a modification within the central part of the peptide (residues 9-28), which is known to be crucial for high affinity binding to CRFBP (10), diminished the affinity of pBF¹¹-rUcn by three orders of magnitude (Table 1, compound 6; FIG. 2). Furthermore, the CRFBP ligand inhibitor h/rCRF⁶⁻³³, its aligned Ucn analog rUcn⁵⁻³², and the respective pBB-analogs were synthesized and tested for their affinity to rCRFBP (Table 1a, compound 7-10; FIG. 3). Table 1b shows the affinity to CRFBP of three further photoactivatable CRF analogs in comparision with the unmodified peptides.

TABLE 1
Biological characterization of CRF-related peptides and their
photoactivatable analogs.

| (1a) | Compound | Peptide | Kₗ [nM] |
|---|---|---|---|
| | 1 | rUcn | 0.23 ± 0.012 |
| | 2 | pBB-rUcn | 0.99 ± 0.124 |
| | 3 | h/rCRF | 0.19 ± 0.009 |
| | 4 | pBB-h/rCRF | 0.13 ± 0.003 |
| | 5 | Diaz-Tyr⁰-h/rCRF | 0.33 ± 0.024 |
| | 6 | pBF¹¹-rUcn | 400 ± 35 |
| | 7 | rUcn⁵⁻³² | 1.1 ± 0.06 |
| | 8 | pBB-rUcn⁵⁻³² | 37 ± 3.5 |
| | 9 | h/rCRF⁵⁻³³ | 0.83 ± 0.026 |
| | 10 | pBB-h/rCRF⁶⁻³³ | 3.4 ± 0.33 |

Inhibition constants (Kₗ) were the mean ± S.E.M. of three independent
experiments performed in duplicate.

| (1b) | Compound | Peptide | IC₅₀ [nM] |
|---|---|---|---|
| | 11 | h/rCRF | 0.54 (0.38-0.71) |
| | 12 | h/rCRF⁶⁻³³ | 1.9 (1.3-2.5) |
| | 13 | pBB-h/rCRF⁶⁻³³ | 3.2 (2.3-4.0) |
| | 14 | [pBPhe³²]h/rCRF⁶⁻³³ | 2.1 (0.70-3.5) |
| | 15 | [pBPhe³²] pBB-h/rCRF⁶⁻³³ | 14 (11-18) |

Immunodetection of the Photoreaction Products

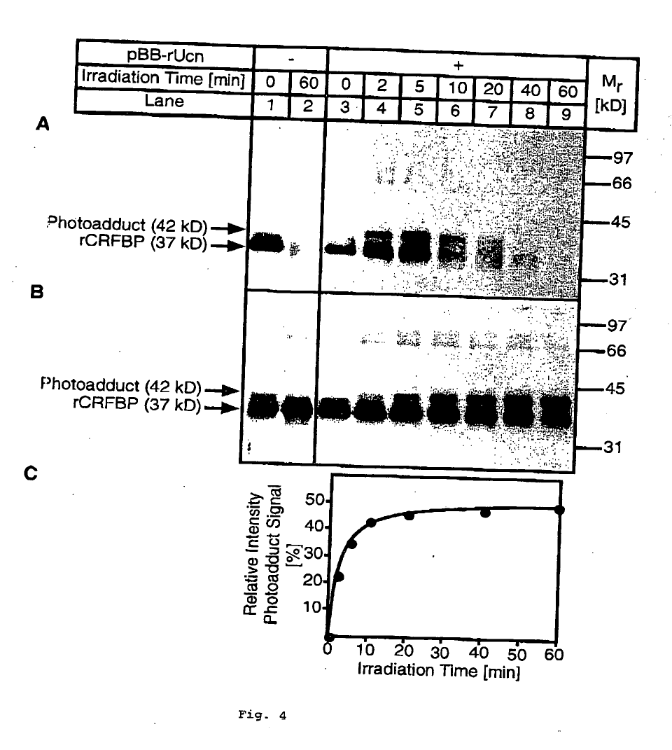

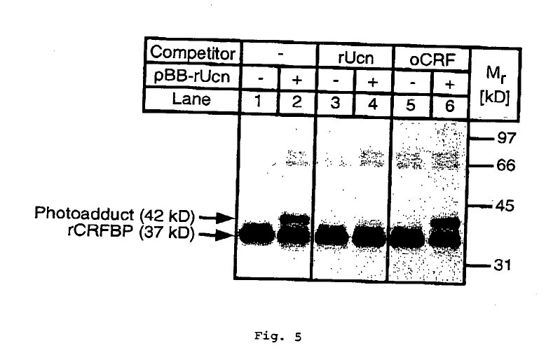

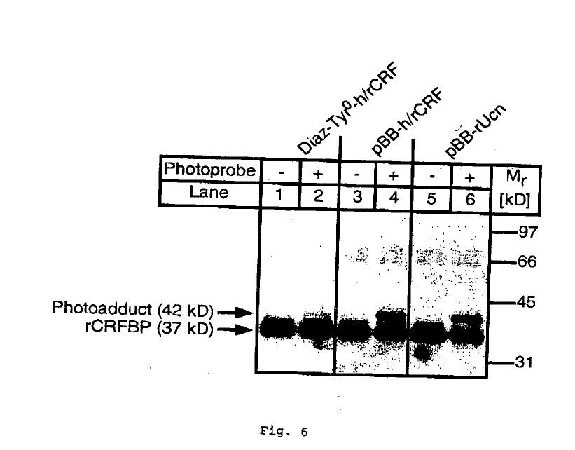

By western blot analysis (FIG. 4, FIG. 5, FIG. 6), rCRFBP produced in HEK 293 cells was detected at an apparent molecular mass of 37 kD which was consistent with reported data for CRFBP (24). Upon irradiation, a photoadduct was identified at the expected apparent molecular mass of 42 kD corresponding to rCRFBP covalently labeled by pBB-rUcn (ΔM_{calc}=4.9 kD). A significant extent of protein degradation was observed when the entire spectrum of the UV lamp was used for irradiation (FIG. 4A). This drawback was overcome by applying the B 270 filter screen that is characterized by a steep transmission cut-off for wavelenghts below 300 nm. Consequently, longer irradiation times became possible resulting in a higher degree of photoincorporation (FIG. 4B). An irradiation time of 30 min was found to be sufficient for maximal photoadduct yields up to 50% (FIG. 4C). In the presence of an excess of rUcn (FIG. 5, lane 4), but not of a low affinity ligand such as oCRF (FIG. 5, lane 6), the formation of the photoadduct was suppressed demonstrating the specificity of the labeling. Furthermore, the photoactivatable high affinity ligands pBB-rUcn, pBB-h/rCRF, and Diaz-Tyr⁰-h/rCRF were compared with respect to their photoadduct yields as determined by immunoblotting. Whereas both benzophenone photoprobes gave an almost identical yield of 50% (FIG. 6, lane 4 and 6), the photoadduct formed by the aryldiazirine photoprobe was hardly detectable (FIG. 6, lane 2).

Purification of the Photoreaction Products and Mass Spectrometric Peptide Mapping of the Photoreaction Products

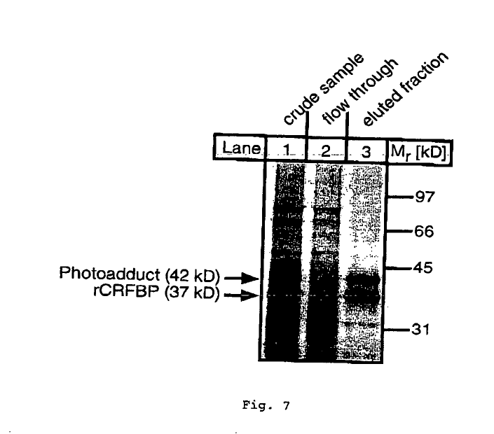

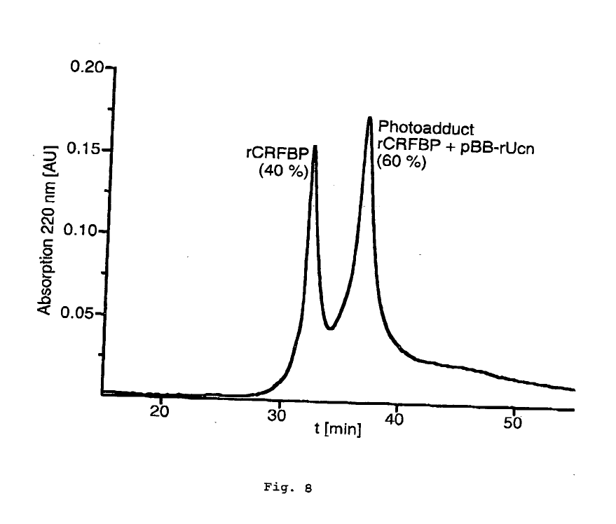

For the purification of the photoreaction products, the PAL experiment was expanded to a preparative scale. Unlabeled binding protein and the photoadduct were co-purified by nickel-affinity chromatography utilizing the C-terminal His₆-sequence fused to rCRFBP and the purified proteins were analyzed by SDS PAGE (FIG. 7). Detection of the photoreaction products by silver staining confirmed the photoadduct yield of 50%. The fraction eluted from the affinity matrix (FIG. 7, lane 3) was analyzed by HPLC (FIG. 8) on-line coupled to the mass spectrometer revealing a significant separation of unlabeled rCRFBP from the photoadduct on the basis of hydrophobicity. The photoadduct yield was found to be 60% as determined by integration of the UV trace (FIG. 8). The molecular masses of rCRFBP (M_{obs}=35746/M_{calc}=35741) and of the photoadduct (M_{obs}=40656/M_{calc}=40657) obtained from the MS signal of this analysis (data not shown) confirmed the expected mass shift for covalent attachment of one molecule pBB-rUcn (ΔM_{obs}=4910/ΔM_{calc}=4916). No significant extent of photodegradation was detected in the mass spectrum of unlabeled rCRFBP or photoadduct, respectively.

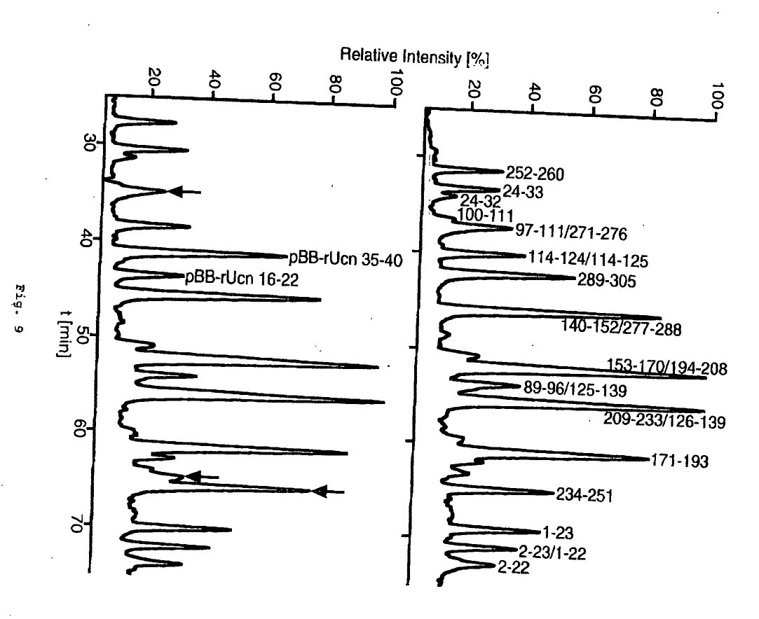

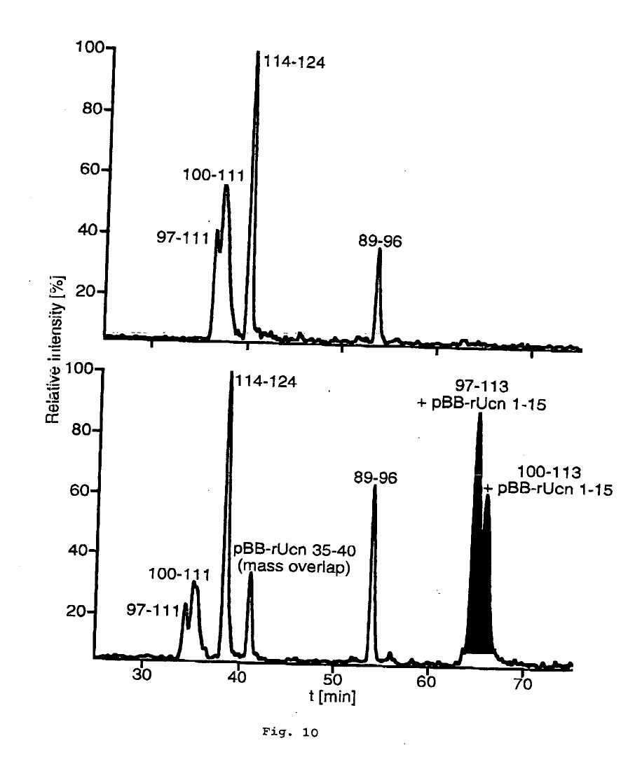

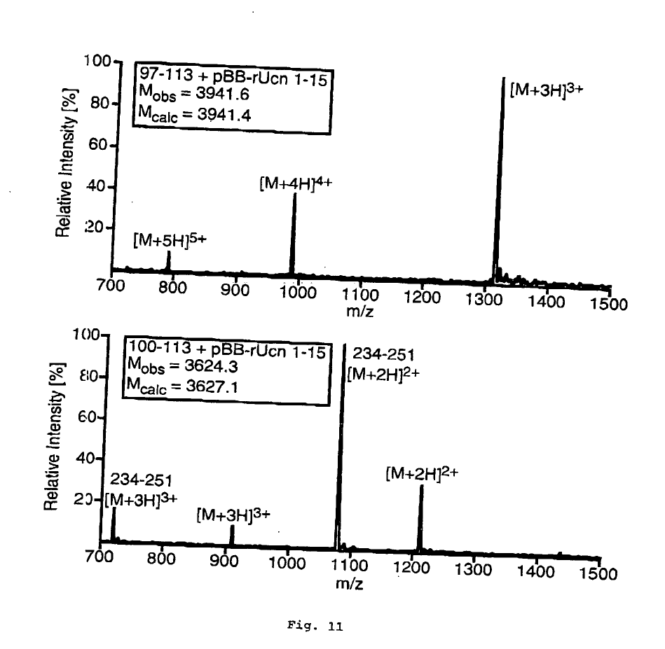

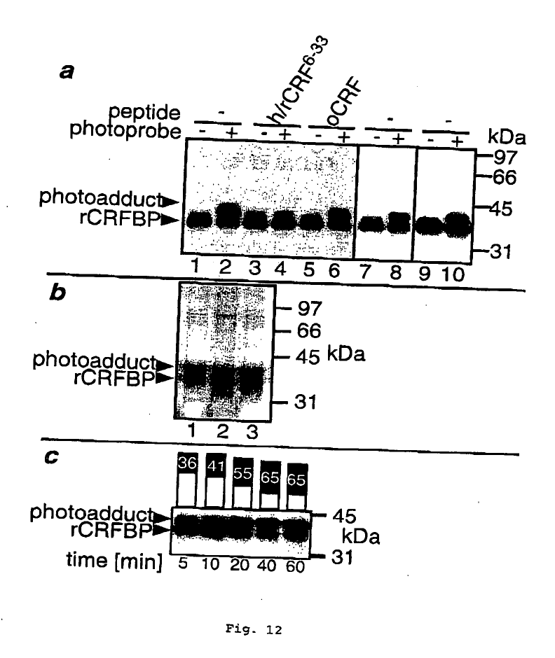

The affinity purified photoreaction products were derivatized by means of S-carboxamidomethylation and separated from each other by RP-HPLC. For the identification of the photolabeled peptides, the photoadduct was subjected to proteolysis using trypsin or a combination of the endoproteinase AspN and trypsin followed by HPLC-MS analysis of the digest mixture. A tryptic digest of the unlabeled rCRFBP was used as control in order to detect alterations in the signal pattern of the obtained chromatograms (FIG. 9). Comparison of the peptide masses derived from the photoadduct with that derived from the unlabeled protein revealed that the abundance of the fragments rCRFBP(97-111) and rCRFBP(100-111) were significantly reduced in the digest of the photoadduct (FIG. 10). Consistently, the peptides rCRFBP(97-113) and rCRFBP(100-113) both labeled with pBB-rUcn¹⁻¹⁵ were identified as the corresponding counterparts indicating that the trypsin cleavage site Arg¹¹¹ was blocked in the photoadduct (FIG. 10, FIG. 11). The remaining amount of rCRFBP(97-111) and rCRFBP(100-111) in the photoadduct digest might be mainly due to incomplete separation of the proteins by RP-HPLC and probably also to a certain extent of unspecific labeling.

The photoactivatable CRFBP ligand inhibitors pBB-h/rCRF⁶⁻³³ and [pBF³²]h/rCRF⁶⁻³³ were treated as described above to identify the photolabeled amino acids with the following results: PBB-h/rCRF⁶⁻³³ reacted with the CRFBP fragment 34-38, an analysis by tandem mass spectrometry showed that only Arg³⁶ was photolabeled. [pBF³²]h/rCRF⁶⁻³³ selectively labeled the CRFBP fragment 12-26, from the complete blocking of the tryptic site it can be followed that Lys²² or Arg²³ is photolabeled.

The above described peptides fulfill all important requirements of a photoprobe to be used for PAL of the CRFBP: high affinity binding, specific labeling, and high photoreaction yields. The obtained photoadduct was stable under the conditions of the applied analytical methods, which was not the case for a photoadduct formed by Diaz-Tyr⁰-oCRF and the N-terminal domain of rCRFR1. Accordingly, the present invention relates to a ligand of the Corticotropin-Releasing Factor (CRF)-binding protein (CRFBP) selected from the group consisting of CRF, Urocortin (Ucn), [Ala²¹]Svg and Urotensin I, said ligand comprising a covalently linked benzophenone moiety.

Theoretical consideration about the physicochemical properties pointed to position 21 of Svg as the amino acid controlling the high affinity binding to CRFBP. On this basis a Svg derivative was synthesized by replacing the Glu²¹ of Svg by alanine ([Ala²¹]Svg). It could be demonstrated that the replacement of Glu²¹ in Svg by Ala increased the affinity to CRFBP by two orders of magnitude (see for example WO 02/24732).

In another embodiment, the present invention relates to functional fragments of the ligand of the Corticotropin-Releasing Factor (CRF)-binding protein (CRFBP) selected from the group consisting of CRF, Urocortin (Ucn), Sauvagine and Urotensin, said ligand comprising a covalently linked benzophenone moiety. In this context the term “functional fragments” refers to biologically active fragments of the ligand as described above exhibiting activity similar, but not necessarily identical, to an activity of said ligands.

A functional fragment based on the sequence of a CRF-like peptide may, e.g. be composed of some of the amino acids of the CRF sequence 9-28. It is envisaged that the affinity should be not more than one order of magnitude lower than that of h/rCRF. In addition, it is envisaged that these fragments show the same ability as h/rCRF to prevent the formation of a photoadduct between CRFBP and pBB-h/rCRF⁶⁻³³ (see for example_compound 10 in Table 1a). The affinity may be determined by using a scintillation proximity assay as described recently (K. Eckart et al. (2001) A single amino acid serves as an affinity switch between the receptor and the binding protein and corticotropin-releasing factor: Implications for the design of agonists and antagonists. Proc. Natl. Acad. Sci. 98: 11142-11147).

In a preferred embodiment, said fragments are selected from a group consisting of [Ala²¹]Svg ⁵⁻³³, Urotensin I ⁶⁻³³,CRF⁶⁻³³ and/or Ucn⁵⁻³².

In accordance with the present invention, the fragments like CRF⁶⁻³³ or Ucn⁵⁻³² as mentioned herein are characterized by the respective position of an amino acid within the respective amino acid sequence of the full-length ligand. Thus, the respective fragment is indicated via the small number exponent which appears next to the abbreviation of the full-length ligand. Thus, e.g. “rUcn⁵⁻³²” relates to a fragment of rUcn which consists of amino acid 5 to 32 of rUcn. Alternatively, the respective amino acid residue is indicated via the respective one-letter code which is also well known in the art. In this respect, e.g. the term “Ala²¹” relates to the amino acid residue alanine at position 21 of the respective amino acid sequence. Furthermore it will be appreciated by the person skilled in the art that the respective numbering of the “position” of an amino acid residue in the context of the present invention is based on the orientation of the underlying peptidic sequence, starting with the N-terminus. Accordingly, position one (1) denotes the N-terminal amino acid residue and so on. The mentioned one-letter and three-letter code of the amino acid residues is, for example, described in Stryer, Biochemistry.

In another embodiment of the ligand of the invention, i.e. a ligand which comprises a covalently linked benzophenone moiety, said benzophenone moiety is covalently linked to the α-amino group of said ligand.

In a preferred embodiment said benzophenone moiety is covalently linked to the α-amino group of said ligand via an N-hydroxysuccinimide ester of said benzophenone moiety. The term “α-amino acid” in the context of the present invention relates to each known natural or synthetic organic compound carrying one amino group and one carboxylate group together on one of its carbon atoms.

The respective covalent linkage may be achieved by means and methods which are exemplified herein, e.g. in the appended examples.

In another preferred embodiment said benzophenone moiety is provided by para-benzoylbenzoic acid or para-hydroxybenzoylbenzoic acid.

In order to minimize changes in the peptides properties by the incorporation of the photoactivatable group, it was considered to replace aromatic or hydrophobic residues by L-para-benzoylphenylalanine. Since we wanted to label the C-terminus of h/rCRF⁶⁻³³ His³² was considered to match best these requirements. In addition, a peptide derived from h/rCRF⁶⁻³³ carrying L-para-benzoylphenylalanine in position 34 of h/rCRF resulting in a C-terminal elongation of h/rCRF⁶⁻³³ showed almost identical affinity and photoadduct yields as observed for the analog labeled in position 32. However, no specifically labeled CRFBP fragment could be identified and this was interpreted in non-specific labeling of CRFBP by the photoreaction. On the basis of this observation and the proposed model for the interaction of h/rCRF⁶⁻³³ with CRFBP, it may be proposed that the amino acid residues of h/rCRF located in the opposite site of the hydrophobic and hydrophilic patches in the helical wheel projection of h/rCRF (K. Eckart et al. (2001) A single amino acid serves as an affinity switch between the receptor and the binding protein and corticotropin-releasing factor: Implications for the design of agonists and antagonists. Proc. Natl. Acad. Sci. 98: 11142-11147) may be best suited for replacement by the photoactivatable amino acid L-para-benzoylphenylalanine. In particular, the residues Val¹⁸, L¹⁴, M²¹, L¹⁰, A²⁸, and A²⁴ may be the best candidates.

The present invention also relates to the ligand CRF⁶⁻³³ wherein said ligand comprises a covalently linked benzophenone moiety wherein the histidine residue at position 32 of said CRF⁶⁻³³ is replaced by L-para-benzoylphenylalanine.

Recently, it was demonstrated that residue 22 of h/rCRF is of special importance for high affinity binding to CRFBP (K. Eckart et al. (2001) A single amino acid serves as an affinity switch between the receptor and the binding protein and corticotropin-releasing factor: Implications for the design of agonists and antagonists. Proc. Natl. Acad. Sci. 98: 11142-11147). Furthermore, the model generated from the results of the photolabelling experiments of CRFBP with the photopeptides derived from h/rCRF⁶⁻³³ indicated, that residue 9 of h/rCRF may be also of importance for high affinity binding to CRFBP (O. Jahn et al. (2002) The binding protein of corticotropin-releasing factor: ligand binding site and subunit structure. Proc. Natl. Acad. Sci. submitted). Therefore, although the authors do not wish to become bound be theory, it may be proposed that a h/rCRF fragment with the sequence of residues 9-22 (h/rCRF⁹⁻²²) or analogues peptides based on Ucn⁸⁻²¹, [A²¹]Svg⁸⁻²¹, and Urotensin I⁹⁻²¹ is sufficient for high affinity binding to CRFBP.

In view of the finding that CRFBP forms a dimer after association with ligand (R J Woods et al. (1994), Endocrinology 135: 768-73) it was investigated whether both photoreaction sites resided in the same CRFBP monomer by employing the bifunctional peptide [pBPhe³²]pBB-h/rCRF⁶⁻³³. By immunoblotting, the photoadduct formed by [pBPhe³²]pBB-h/rCRF⁶⁻³³ was exclusively detected at an apparent molecular mass of 41 kD indicating that no covalent crosslinking of the monomers occurred. It was concluded on the basis of these experiments that CRF bound to one subunit of the proposed CRFBP complex.

Thus, it is also envisaged that the ligands of the present invention are coupled to at least one benzophenone moiety as defined herein. In particular, it is also envisaged that the ligands of the present invention are coupled with two, three, four or five benzophenone moieties. It is, therefore, to be understood that the present invention also encompasses ligands containing benzophenone moieties at different positions as indicated herein.

In a preferred embodiment of the above mentioned ligand CRF⁶⁻³³ (i.e. the ligand CRF⁶⁻³³ wherein said ligand comprises a covalently linked benzophenone moiety wherein the histidine residue at position 32 of said CRF⁶⁻³³ is replaced by L-para-benzoylphenylalanine), para-benzoylbenzoic acid or para-hydroxybenzoylbenzoic acid is covalently linked to the α-amino group of said ligand.

It is also an objective of the present invention to provide the above mentioned ligands, wherein these ligands further comprise at the N-terminus a tyrosine residue to which said benzophenone moiety is covalently linked.

The development of new photoprobes generates new perspectives in the research of CRF biology. Besides their application as powerful tools for the characterization of the binding sites of CRFBP and CRFR, the new photoprobes might be also useful for the investigation of the cell biological fate of CRFBP and CRFR after ligand binding. Therefore, an additional detection-label must be incorporated into the photoactivatable ligands. This may be achieved analogous to Diaz-Tyr⁰-h/rCRF by N-terminal elongation of the peptides with a Tyr residue which is amenable to radiolabeling with iodine. Furthermore, for benzophenone-based photoprobes the para-hydroxybenzoylbenzoyl-group can be used instead of the pBB-group as described (29). Thereby, the photoreactive group can be directly radioiodinated without the need of an additional Tyr residue.

Thus, the person skilled in the art is well aware that it is also envisaged to label the ligands of the invention with an appropriate marker or tag for specific applications, such as for the detection of the presence of CRFBP and or the CRF-receptors in a sample derived from an organism, in particular mammals, preferably human. A number of companies such as Pharmacia Biotech (Piscataway N.J.), Promega (Madison Wis.), and US Biochemical Corp (Cleveland Ohio) supply commercial kits and protocols for these procedures. Suitable reporter molecules or labels include radionuclides such as iodine (¹²⁵I, ¹²¹I), carbon (¹⁴C), sulfur (³⁵S), tritium (³H), indium (¹¹²In), and technetium (⁹⁹mTc), and fluorescent labels, such as fluorescein and rhodamine, and biotin, enzymes (like horse radish peroxidase, β-galactosidase, alkaline phosphatase), chemi- or bioluminescent compounds (like dioxetanes, luminol or acridiniums), fluorochromes (like fluorescein, rhodamine, Texas Red, etc.) or chromogenic agents as well as substrates, cofactors, inhibitors, magnetic particles and the like. Patents teaching the use of such labels include US patents U.S. Pat. No. 3,817,837; U.S. Pat. No. 3,850,752; U.S. Pat. No. 3,939,350; U.S. Pat. No. 3,996,345; U.S. Pat. No. 4,227,437; U.S. Pat. No. 4,275,149 and U.S. Pat. No. 4,366,241. It is also envisaged that said tag is selected, but not limited to, from the group consisting of His-tag, Streptavidin-tag, HA-tag, GST-tag, CBP-tag, MBP-tag, FLAG-tag, myc as well as single-chain fragments (sc Fvs) of antibody binding regions. A variety of techniques are available for labeling biomolecules, are well known to the person skilled in the art and are considered to be within the scope of the present invention. Such techniques are, e.g., described in Tijssen, “Practice and theory of enzyme immuno assays”, Burden, R H and von Knippenburg (Eds), Volume 15 (1985), “Basic methods in molecular biology”; Davis L G, Dibmer M D; Battey Elsevier (1990), Mayer et al., (Eds) “Immunochemical methods in cell and molecular biology” Academic Press, London (1987), or in the series “Methods in Enzymology”, Academic Press, Inc. It is also envisaged that the respective label might also be coupled to the ligands of the invention via short peptidic or non-peptidic spacer-molecules which are well known in the art and encompass e.g. Glycin, 1,6-Diaminohexan and/or short fragments of not more than 5 continued amino acids derived from h/rCRF (e.g. h/rCRF¹⁻⁵).

There are many different labels and methods of labeling known to those of ordinary skill in the art. Labeling procedures, like covalent coupling of enzymes or biotinyl groups, iodinations, phosphorylations, biotinylations, are well known in the art. Detection methods comprise, but are not limited to, autoradiography, fluorescence microscopy, direct and indirect enzymatic reactions, FACS-analysis etc.

Accordingly, the present invention relates to the ligands of the invention which are labeled.

The person skilled in the art is well aware that such a label should have no or only a minor effect on the binding characteristics of the ligand, i.e. as already indicated above it is envisaged that a labeled ligand retains a high affinity to its respective receptor (either CRFR or CRFBP). The same holds true for the coupling of the ligands of the invention to more than one benzophenone moiety and/or to further moieties as indicated herein. The skilled person is aware of methods of how to measure such binding characteristics which are known in the art and which have already been mentioned before. In addition, the present invention discloses suitable techniques in the appended examples which guide the skilled person when designing such a suitable test. It is envisaged that coupling a label, another moiety and/or a further benzophenone moiety as described herein to ligands of the invention reduces the binding affinity of said labeled or otherwise altered ligand to its respective receptor not more than 30%, not more than 25% not more than 20%, not more than 15%, not more than 10%, not more than 5% and/or not more than 0,1%.

The binding affinity to CRFBP of CRFR may be determined in competition experiments employing different concentrations of cold (i.e. non-radioactive) ligand and a constant concentration of radioactive ligand. The IC₅₀ value has to be determined by curve fitting algorithms as e.g. used in the computer program Prism (GraphPad Software). The affinity of a ligand labelled with a benzophenone moiety should be not decreased by more than one order of magnitude. This means in our test system an IC₅₀ cut-off of approximately 10 nM. Thus, the binding affinity of such altered ligands of the invention can easily be determined as demonstrated herein above as well as in the appended examples.

In a preferred embodiment said tyrosine residue at the N-terminus to which said benzophenone moiety is covalently linked, is labeled.

In an even more preferred embodiment said tyrosine residue is radioactively labeled.

In a most preferred embodiment said tyrosine residue is labeled with ¹²⁵I.

In a preferred embodiment, it is envisaged that the ligand of the present invention is characterized by one or a combination or all of the following characteristics:

a) it binds to a CRFBP wherein said CRFBP is selected from the group consisting of rat CRFBP (rCRFBP; accession number P24388), human CRFBP (hCRFBP; accession number P24387), murine CRFBP (mCRFBP; accession number Q60571), sheep CRFBP (accession number Q28557) and/or CRFBP of Xenopus laevis (accession number Q91653);

b) it binds with high affinity to said CRFBP (IC₅₀<10 nM);

c) it does not show specific binding to CRFR1 which is defined as no detectable competition with radiolabeled h/rCRF in concentrations up to 3 μM; and

d) it does not show specific binding to CRFR2 which is defined as no detectable competition with radiolabeled Sauvagine in concentrations up to 3 μM.

Thus in accordance with the above, the ligand of the invention is a ligand of CRFBP wherein said CRFBP within the meaning of the present invention is selected from the group consisting of rat CRFBP (rCRFBP), human CRFBP (hCRFBP), murine CRFBP (mCRFBP), sheep CRFBP and/or CRFBP of Xenopus laevis.

In a preferred embodiment of the ligand of the invention said CRF is human/rat CRF (h/rCRF), murine CRF, porcine CRF, bovine CRF Tilipia CRF, frog CRF, sucker CRF, sockey salmon CRF or sockey salmon CRF and/or said CRF⁶⁻³³ is human/rat CRF⁶⁻³³ (h/r CRF⁶⁻³³), murine CRF⁶⁻³³, porcine CRF⁶⁻³³, bovine CRF⁶⁻³³ Tilipia CRF⁶⁻³³, frog CRF⁶⁻³³, sucker CRF⁶⁻³³, or sockey salmon CRF⁶⁻³³.

In another preferred embodiment of the ligand of the invention said Ucn is ratUcn (rUcn), human Ucn murine Ucn, ovine Ucn or hamster Ucn.

In another embodiment of the ligand of the invention said ligand is fused to another moiety such as a tag, a heterologous protein and/or a label as indicated herein. For example the ligands of the invention may be fused with or coupled to another compound, such as a compound to increase the stability and/or solubility of the ligand (for example, polyethylene glycol), or fusion of the ligand with additional amino acids, such as an IgG Fc fusion region peptide, or leader or secretary sequence, or a sequence facilitating purification. Moreover, the ligands of the invention can be fused to marker sequences, such as a peptide which facilitates purification of such ligands. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz, Proc. Natl. Acad. Sci. USA 86 (1989); 821-824, for instance, hexa-histidine provides for convenient purification of the fusion protein. Another peptide tag useful for purification, the “HA” tag, corresponds to an epitope derived from the influenza hemagglutinin protein; see Wilson, Cell 37 (1984); 767. Such variant ligands are deemed to be within the scope of those skilled in the art from the teachings herein. It is also envisaged that such moiety such as a tag, a heterologous protein and/or a label are coupled to the ligand of the invention with the use of spacer molecules as mentioned herein before.

The invention also refers to a process for the purification of a CRFBP which comprises reacting said CRFBP with the ligand of the invention, performing photoaffinity labeling, and purifying the resultant photoreaction products. Said purification can be achieved by means and methods which are well known to the skilled person and, further, which are exemplified herein. For example, it is envisaged that such a purification can be achieved by HPLC, FPLC, Immunoprecipitation, affinity purification, ultrafiltration, gel electrophoresis, isolelectric focusing and or (preparative) ultracentrifugation.

In a preferred embodiment said purification is achieved by HPLC as demonstrated in the appended examples.

The present invention also relates to a process for the characterization of the binding site of a CRFBP which comprises purifying the CRFBP according to the process described herein, fragmenting the purified product and determining the amino acid sequence of the relevant fragment. In this context, it is to be understood that the purification, the fragmenting of the purified product as well as the determination of the amino acid sequence can easily be achieved by means and methods which are well known to the skilled person and which are also exemplified in the appended examples.

Peptides such as pBB-h/rCRF⁶⁻³³ (10) may be used to detect CRFBP in various tissues such as brain tissues (hippocampus and cortex), placenta and liver. The CRFBP antibody recently described (O. Jahn et al. (2000) Pharmacological characterization of recombinant rat corticotropin releasing factor binding protein using different sauvagine analogs. Peptides, 22:47-56) showed severe crossreactivity with other proteins when used in brain slices and could be therefore not be used for this purpose. A covalent complex between CRFBP and a benzophenone labeled CRF-like peptide may be detected by using radioactive labeling of the ligand or by using the commercially available CRF or Ucn antibody as described (O. Jahn et al. (2002) The binding protein of corticotropin-releasing factor: ligand binding site and subunit structure. Proc. Natl. Acad. Sci. submitted). Another choice would be the detection of this complex by employing a Gd chelating agent coupled to CRF with NMR.

Thus, it is also an object of the present invention to use the ligands of the present invention for detecting CRFBP. Such a method is described above and also exemplified in the appended examples.

In a preferred embodiment said detection as mentioned herein above is performed in the presence of CRF receptors. pBB-h/rCRF6-33 (10) provides the possibility to detect CRFBP specifically in the presence of CRFR. In the tissues containing CRFBP also CRFR subtypes are co-localized. Therefore, peptides binding specifically to CRFBP are needed for the specific detection of CRFBP.

In another preferred embodiment of the detection method of the invention, said CRFBP is detected in a biological fluid. The term “biological fluid” within the context of the present invention relates to blood, serum, plasma and or cerebrospinal fluids. In a preferred embodiment, said fluids are derived from mammalian animals, preferably from humans.

It is another object of the present invention to use the ligand of the invention for identifying the binding site in CRFBP of a CRF or a CRF-like peptide.

The present invention also relates to a kit which comprises at least one of the ligands of the invention.

In a preferred embodiment, the present invention also relates to a diagnostic kit comprising the ligands of the invention. In addition, it is also envisaged that the ligands of the invention are comprised in a diagnostic composition. The components of the diagnostic composition and/or the kit of the invention may be packaged in containers such as vials, optionally in buffers and/or solutions. If appropriate, one or more of said components may be packaged in one and the same container. The parts of the kit of the invention can also be packaged individually in vials or in combination in containers or multicontainer units. Additionally or alternatively, one or more of said components may be adsorbed to solid support such as, e.g., a nitrocellulose filter or nylon membrane, or to the well of a microtiter plate. Solid phases are known to those in the art and may comprise polystyrene beads, latex beads, magnetic beads, colloid metal particles, glass and/or silicon chips and surfaces, nitrocellulose strips, membranes, sheets, animal red blood cells, or red blood cell ghosts, duracytes and the walls of wells of a reaction tray, plastic tubes or other test tubes. Suitable methods of immobilizing nucleic acids, (poly)peptides, proteins, antibodies, etc. on solid phases include but are not limited to ionic, hydrophobic, covalent interactions and the like.

The kit of the invention may advantageously be used for carrying out any one of the methods of the invention and could be, inter alia, employed in a variety of applications referred to herein, e.g., in the diagnostic field or as research tool. Manufacture of the kit follows preferably standard procedures which are known to the person skilled in the art.

In a further aspect the present invention relates to a method for identifying an inhibitor of the binding of the ligands of the invention to CRFBP comprising:

(a) contacting a ligand of the invention and a CRFBP as mentioned herein with a compound or a plurality of compounds to be screened;

(b) irradiating the mixture described in (a) with UV light above 300 nm including a significant emission at about 360 nm for effective excitation of the benzophenon photophore; and

(c) determining whether the compound or said plurality of compounds effects an inhibitory effect on the binding of said ligand and said CRFBP.

The term “including a significant emission at about 360 nm” denotes that the emission at 360 nm is not less than the emission of a neighbored wave-length between 320 nm and 380 nm. In a preferred embodiment, said emission is at 360 nm.

The assay can simply be carried out using CRFBP as a cell-free preparation, e.g. affixed to a solid support or in solution. The assay may also simply comprise the steps of mixing a candidate compound or a plurality of compounds with a solution containing the ligand of the invention and CRFBP; measuring binding of the ligand to CRFBP after a suitable photoactivation, and comparing the binding of CRFBP and the ligand of the invention with the binding to a standard. A “suitable photoactivation” denotes the efficient formation of a diradical of the carbonyl group of the benzophenone moiety representing the reactive state of the benzophenone photophore, which is also demonstrated in the appended examples. Further, a suitable “standard” within the meaning of the present invention, in particular in the context of the screening methods as described in the present invention is represented by a reference experiment using 1 nM recombinant rCRFBP, 100 nM pBB-h/rCRF⁶⁻³³ (10), and 1000 nm h/rCRF³. When rCRFBP and pBB-h/rCRF⁶⁻³³ are incubated alone a minimum of 50% photoadduct yield must be detected after 60 minutes irradiation by using SDS-PAGE in combination with Western blotting and immuno detection. The formation of the photoadduct must be inhibited when the same experiment is performed in parallel including h/rCRF⁶⁻³³.

All of these above assays can be used as diagnostic or prognostic markers. The molecules discovered using these assays can be used to treat disease or to bring about a particular result in a patient (e.g., increase the level of unbound CRF in the cerebrospinal fluid) by inhibiting the CRF/CRFBP-binding.

It is to be understood that the screened compounds as obtainable by the methods of the present invention should have only a low or now affinity to the CRF-receptors CRFR1/2 as mentioned herein below. Therefore, the invention also relates to a method of identifying compounds which inhibit the binding of CRFBP to the ligands of the invention comprising the steps of:

(a) contacting a ligand of the invention and a CRFBP as mentioned herein with a compound or a plurality of compounds to be screened;

(b) irradiating the mixture described in (a) with UV light above 300 nm including a significant emission at about 360 nm for effective excitation of the benzophenone photophore;

(c) determining whether the compound or said plurality of compounds effects an inhibitory effect on the binding of said ligand and said CRFBP; and

(d) determining whether said compound or said plurality of compounds is able to bind to CRFR1 and/or CRFR2; wherein no or low binding to CRFR1 and/or CRFR2 indicates an inhibitor within the meaning of the present invention.

CRFR1 and/or CRFR2 may be obtained from stably transfected imortilized cells (i.e. HEK 293). The cDNA contruct for transfection may contain a strong viral expression promotor (i.e. PCDNA III). Membrane fragments of such cells are obtained after harvesting the cells and can be stored for longer peroids at −80 degree celsius. These methods were already described (S. Sydow et al. STRUCTURE-FUNCTION RELATIONSHIP OF DIFFERENT DOMAINS OF THE RAT CORTICOTROPIN-RELEASING FACTOR RECEPTOR. Molecular Brain Research. 52:182-193).

The term “inhibitor” means in accordance with the present invention a compound or a plurality of compounds capable of interfering with, such as suppressing the binding of CRF to CRFBP i.e. interaction of CRFBP with its corresponding ligand, which may, for example be a h/rCRF. In accordance with the present invention said inhibitor preferably interacts with the ligand, for example by specifically binding to said ligand. “Specifically binding” means “specifically interacting with” whereby said interaction may be, inter alia, covalently, non-covalently and/or hydrophobic. Thus, an inhibitor which may be an antagonist may be a compound which inhibits or decreases e.g. the interaction between a protein and another molecule. Such inhibitors may be obtained by methods described herein. The inhibitors of the present invention preferably have a binding affinity to CRFBP as mentioned herein of at least 10⁵ M⁻¹, preferably higher than 10⁷ M⁻¹ and advantageously up to 10¹⁰ M⁻¹. Even higher bonding affinities are not excluded form the invention.

It is envisaged that the inhibitors of the present invention are capable of a suppression or inhibition of the CRF/CRFBP signaling pathway i.e. they may directly interfere with the CRF/CRFBP signaling pathway/cascade in a cell/subject in a way which is sufficient to suppress the CRF/CRFBP signaling pathway/cascade to at least about 50% as compared to the natural state of said cell/subject. Preferably said inhibition efficiency is at least 80%, 85%, 90% or 95%. In a most preferred embodiment said inhibition rate is 100%.

In a more preferred embodiment said inhibitor as screened or isolated by the methods of the invention as described herein before, has preferably a low or even no affinity to the CRF-receptors like CRFR1 and/or CRFR2. The term “low affinity” in this context means that the binding affinity of said inhibitor as screened and, optionally further improved by the methods described herein below, to the respective CRF-receptors CRFR1 and/or CRFR2 is lower than 30%, lower than 25% lower than 20%, lower than 15%, lower than 10%, lower than 5% and/or lower than 0,1% as compared to a suitable standard ligand of said receptor. A suitable standard ligand to the CRFR1 and/or CRFR2 is for example CRF like h/rCRF, Urocortin, Sauvagine and/or Urotensin I. Accordingly, it will be appreciated by the person skilled in the art that the selectivity of such a compound can be easily determined by using the SPA assay systems described for CRFR1 and CRFR2 as well as CRFBP (K. Eckart et al. (2001) A single amino acid serves as an affinity switch between the receptor and the binding protein and corticotropin-releasing factor: Implications for the design of agonists and antagonists. Proc. Natl. Acad. Sci. 98: 11142-11147). These assay system utilize a SPA system and were adapted to 96 well technology. Therefore, the testing is fast and reliable.

The term “plurality of compounds” is to be understood as a plurality of substances which may or may not be identical. The plurality of compounds may preferably act additively or synergistically. Said compound or plurality of compounds may be chemically synthesized or microbiologically produced and/or comprised in, for example, samples, e.g., cell extracts from, e.g., plants, animals or microorganisms. Furthermore, said compound(s) may be known in the art but hitherto not known to be capable of suppressing the CRF/CRFBP pathway. Suitable set ups for the method of the invention are known to the person skilled in the art and are, for example, generally described in Alberts et al., Molecular Biology of the Cell, third edition (1994) and in the appended examples. The plurality of compounds may be, e.g., added to the reaction mixture or a culture medium.

If a sample containing a compound or a plurality of compounds is identified in the method of the invention, then it is either possible to isolate the compound from the original sample identified as containing the compound capable of suppressing the interaction of CRF/CRFBP as mentioned herein before, or one can further subdivide the original sample, for example, if it consists of a plurality of different compounds, so as to reduce the number of different substances per sample and repeat the method with the subdivisions of the original sample. Depending on the complexity of the samples, the steps described above can be performed several times, preferably until the sample identified according to the method of the invention only comprises a limited number of or only one substance(s). Preferably said sample comprises substances of similar chemical and/or physical properties, and most preferably said substances are identical. Various sources for the basic structure of such an inhibitor can be employed and comprise, for example, mimetic analogs of the ligands of the invention. Mimetic analogs of the ligand of the invention or functional fragments thereof can be generated by, for example, substituting the amino acids that are expected to be essential for the biological activity with, e.g., stereoisomers, i.e. D-amino acids; see e.g., Tsukida, J. Med. Chem. 40 (1997), 3534-3541. Furthermore, in case functional fragments are used for the design of biologically active analogs pro-mimetic components can be incorporated into a peptide to reestablish at least some of the conformational properties that may have been lost upon removal of part of the original ligand; see, e.g., Nachman, Regul. Pept. 57 (1995), 359-370.

Furthermore, the ligand of the invention can be used to identify synthetic chemical peptide mimetics that bind to or can function as a ligand, substrate, binding partner or the receptor of the polypeptide of the invention as effectively as does the natural polypeptide; see, e.g., Engleman, J. Clin. Invest. 99 (1997), 2284-2292. In addition, folding simulations and computer redesign of structural motifs of the ligand of the invention can be performed using appropriate computer programs (Olszewski, Proteins 25 (1996), 286-299; Hoffman, Comput. Appl. Biosci. 11 (1995), 675-679). Computer modeling of protein folding can be used for the conformational and energetic analysis of detailed peptide and protein models (Monge, J. Mol. Biol. 247 (1995), 995-1012; Renouf, Adv. Exp. Med. Biol. 376 (1995), 3745). In particular, the appropriate programs can be used for the identification of interactive sites of the ligand and its receptor i.e. CRFBP and/or CRFR1/2, or other interacting proteins by computer assistant searches for complementary peptide sequences (Fassina, Immunomethods 5 (1994), 114-120. Further appropriate computer systems for the design of protein and peptides are described in the prior art, for example in Berry, Biochem. Soc. Trans. 22 (1994), 1033-1036; Wodak, Ann. N.Y. Acad. Sci. 501 (1987), 1-13; Pabo, Biochemistry 25 (1986), 5987-5991. The results obtained from the above-described computer analysis can be used for, e.g., the preparation of peptide mimetics of the ligand of the invention or functional fragments thereof. Such pseudopeptide analogues of the amino acid sequence of the ligand of the invention may very efficiently mimic the parent ligand (Benkirane, J. Biol. Chem. 271 (1996), 33218-33224). For example, incorporation of easily available achiral ω-amino acid residues into a ligand of the invention or a functional fragment thereof results in the substitution of amide bonds by polymethylene units of an aliphatic chain, thereby providing a convenient strategy for constructing a peptide mimetic (Banerjee, Biopolymers 39 (1996), 769-777). Superactive peptidomimetic analogues of small peptide hormones in other systems are described in the prior art (Zhang, Biochem. Biophys. Res. Commun. 224 (1996), 327-331). Appropriate peptide mimetics of, the ligand of the present invention can also be identified by the synthesis of peptide mimetic combinatorial libraries through successive amide alkylation and testing the resulting compounds, e.g., for their binding properties as mentioned herein. Methods for the generation and use of peptidomimetic combinatorial libraries are described in the prior art, for example in Ostresh, Methods in Enzymology 267 (1996), 220-234 and Dorner, Bioorg. Med. Chem. 4 (1996), 709-715. Furthermore, a three-dimensional and/or crystallographic structure of the ligand of the invention either alone or in combination with CRFBP, CRFR1 and/or CRFR2 can be used for the design of peptide mimetic inhibitors of the CRFBP-binding activity of the ligand of the invention (Rose, Biochemistry 35 (1996), 12933-12944; Rutenber, Bioorg. Med. Chem. 4 (1996), 1545-1558).

It is also well known to the person skilled in the art, that it is possible to design, synthesize and evaluate mimetics of small organic compounds that, for example, inhibit the interaction of CRFBP and the ligand of the invention (as mentioned herein before). The compounds which can be tested and identified according to a method of the invention may be expression libraries, e.g., cDNA expression libraries, peptides, proteins, nucleic acids, antibodies, small organic compounds, hormones, peptidomimetics, PNAs or the like (Milner, Nature Medicine 1 (1995), 879-880; Hupp, Cell 83 (1995), 237-245; Gibbs, Cell 79 (1994), 193-198 and references cited supra).

The compounds isolated by the above methods also serve as lead compounds for the development of analog compounds. The analogs should have a stabilized electronic configuration and molecular conformation that allows key functional groups to be presented to the ligand or its receptor CRFBP, CRFR1 and/or CRFR2 in substantially the same way as the lead compound. In particular, the analog compounds have spatial electronic properties which are comparable to the binding region, but can be smaller molecules than the lead compound, frequently having a molecular weight below about 2 kD and preferably below about 1 kD. Identification of analog compounds can be performed through use of techniques such as self-consistent field (SCF) analysis, configuration interaction (CI) analysis, and normal mode dynamics analysis. Computer programs for implementing these techniques are available; e.g., Rein, Computer-Assisted Modeling of Receptor-Ligand Interactions (Alan Liss, New York, 1989). Methods for the preparation of chemical derivatives and analogues are well known to those skilled in the art and are described in, for example, Beilstein, Handbook of Organic Chemistry, Springer edition New York Inc., 175 Fifth Avenue, New York, N.Y. 10010 U.S.A. and Organic Synthesis, Wiley, New York, USA. Furthermore, said derivatives and analogues can be tested for their effects according to methods known in the art; see also supra.

Furthermore, peptidomimetics and/or computer aided design of appropriate derivatives and analogues can be used, for example, according to the methods described above.

The invention further relates to a method of modifying an inhibitor obtained by the methods of the invention as a lead compound to achieve (i) modified site of action, spectrum of activity, organ specificity, and/or (ii) decreased toxicity (improved therapeutic index), and/or (ill) decreased side effects, and/or (iv) modified onset of therapeutic action, duration of effect, and/or (v) modified pharmakinetic parameters (resorption, distribution, metabolism and excretion), and/or (vi) modified physico-chemical parameters (hygroscopicity, color, taste, odor, stability, state), and/or (vii) improved general specificity, organ/tissue specificity, and/or (viii) optimized application form and route by (i) esterification of carboxyl groups, or (ii) esterification of hydroxyl groups with carbon acids, or (iii) esterification of hydroxyl groups to, e.g. phosphates, pyrophosphates or sulfates or hemi succinates, or (iv) formation of pharmaceutically acceptable salts, or (v) formation of pharmaceutically acceptable complexes, or (vi) synthesis of pharmacologically active polymers, or (vii) introduction of hydrophilic moieties, or (viii) introduction/exchange of substituents on aromates or side chains, change of substituent pattern, or (ix) modification by introduction of isosteric or bioisosteric moieties, or (x) synthesis of homologous compounds, or (xi) introduction of branched side chains, or (xii) conversion of alkyl substituents to cyclic analogues, or (xiii) derivatisation of hydroxyl group to ketales, acetales, or (xiv) N-acetylation to amides, phenylcarbamates, or (xv) synthesis of Mannich bases, imines, or (xvi) transformation of ketones or aldehydes to Schiff's bases, oximes, acetales, ketales, enolesters, oxazolidines, thiozolidines or combinations thereof.

The various steps recited above are generally known in the art. They include or rely on quantitative structure-action relationship (QSAR) analyses (Kubinyi, 1993), combinatorial biochemistry, classical chemistry and others (see, for example, Holzgrabe and Bechtold, 2000).

Once the described compound has been identified and obtained, it is preferably provided in a therapeutically acceptable form. Thus, the present invention also relates to a method of producing a therapeutic agent comprising the steps of the methods of the invention described above; and

(i) synthesizing the compound obtained or identified or an analog or derivative thereof in an amount sufficient to provide said agent in a therapeutically effective amount to a patient; and/or

(ii) combining the compound obtained or identified or an analog or derivative thereof with a pharmaceutically acceptable carrier.

In summary, the present invention provides methods for identifying compounds which are capable of modulating the CRF/CRFBP pathway due to their direct or indirect inhibition of CRF and CRFBP. Accordingly compounds identified in accordance with the method of the present invention to be inhibitors of the CRF/CRFBP binding are also within the scope of the present invention.

Compounds found to inhibit CRF/CRFBP-binding, i.e. enhance the concentration of unbound CRF, may be used in the treatment of cognitive deficits such as Alzheimers disease. In addition, it may be also possible to increase the level of CRF in hippocampal and cortical tissues by such compounds without producing anxiety like effects as observed when i.e. mice were injected with CRF into the brain.

The compounds identified or obtained according to the method of the present invention are thus expected to be very useful in diagnostic and in particular for therapeutic applications. In a further embodiment the invention relates to a method for the production of a pharmaceutical composition comprising the steps of any one of the above described methods and formulating the compound drug candidate identified or a derivative or homologue thereof in a pharmaceutically acceptable form. The therapeutically useful compounds identified according to the method of the invention may be formulated and administered to a patient as discussed above. For uses and therapeutic doses determined to be appropriate by one skilled in the art; see infra. Furthermore, the present invention relates to a method for the preparation of a pharmaceutical composition comprising the steps of the above-described methods; and formulating a drug or pro-drug in the form suitable for therapeutic application.

Thus, in a further embodiment the invention relates to a method for the production of a pharmaceutical composition comprising formulating and optionally synthesizing the inhibitor identified in the above described method of the invention in a pharmaceutically acceptable form. Hence, the present invention generally relates to a method of making a therapeutic agent comprising synthesizing the inhibitors according to the invention in an amount sufficient to provide said agent in a therapeutically effective amount to the patient. Methods for synthesizing these agents are well known in the art and are described, e.g. above.

Examples of suitable pharmaceutical carriers are well known in the art and include phosphate buffered saline solutions, water, emulsions, such as oil/water emulsions, various types of wetting agents, sterile solutions etc. Compositions comprising such carriers can be formulated by well known conventional methods. These pharmaceutical compositions can be administered to the subject at a suitable dose. Administration of the suitable compositions may be effected by different ways, e.g., by intravenous, intraperitoneal, subcutaneous, intramuscular, topical or intradermal administration. The dosage regimen will be determined by the attending physician and clinical factors. As is well known in the medical arts, dosages for any one patient depends upon many factors, including the patient's size, body surface area, age, the particular compound to be administered, sex, time and route of administration, general health, and other drugs being administered concurrently. The compositions of the invention may be administered locally or systemically. Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like. Furthermore, the pharmaceutical composition of the invention may comprise further agents depending on the intended use of the pharmaceutical composition.

Drugs or pro-drugs after their in vivo administration are metabolized in order to be eliminated either by excretion or by metabolism to one or more active or inactive metabolites (Meyer, J. Pharmacokinet. Biopharm. 24 (1996), 449-459). Thus, rather than using the actual compound or drug identified and obtained in accordance with the methods of the present invention a corresponding formulation as a pro-drug can be used which is converted into its active in the patient. Precautionary measures that may be taken for the application of pro-drugs and drugs are described in the literature; see, for review, Ozama, J. Toxicol. Sci. 21 (1996), 323-329.

In a preferred embodiment of the method of the present invention said drug or prodrug is a derivative of a medicament as defined herein before.

The therapeutically useful compounds identified according to the method of the invention may be administered to a patient by any appropriate method for the particular compound, e.g., orally, intravenously, parenterally, transdermally, transmucosally, or by surgery or implantation (e.g., with the compound being in the form of a solid or semi-solid biologically compatible and resorbable matrix) at or near the site where the effect of the compound is desired. Therapeutic doses are determined to be appropriate by one skilled in the art, see also infra.

The figures show:

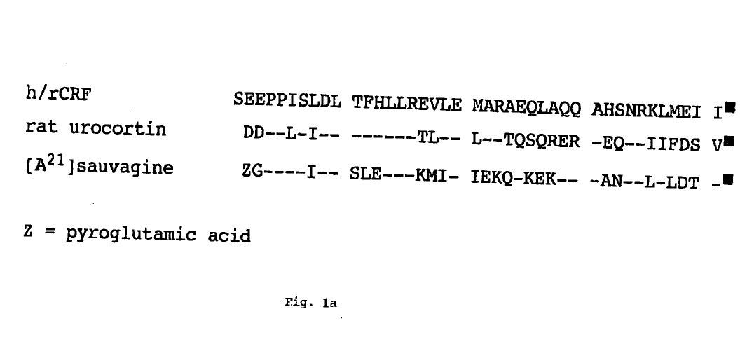

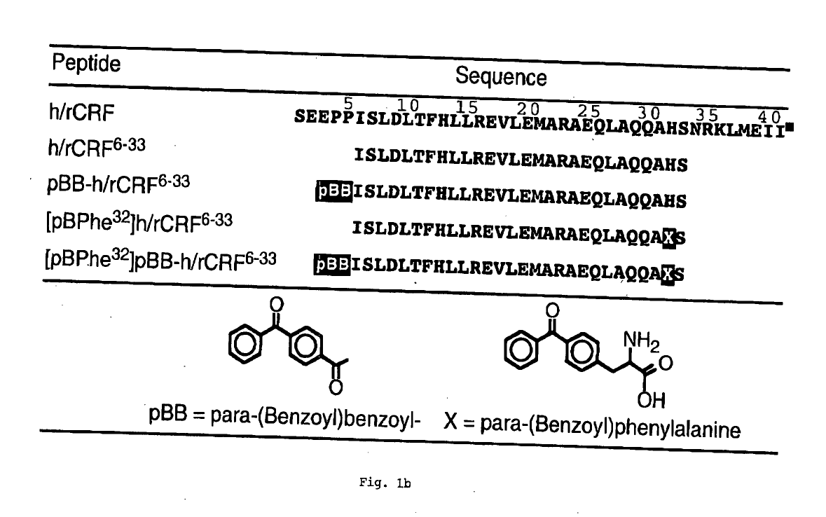

FIG. 1: Amino acid sequences of some peptides of the invention:

1a: Amino acid sequence alignment of h/rCRF, rUcn and [A²¹]-Sauvagine. Identical amino acids are indicated by dashes. ▪=C-terminal amide.

1b: Amino acids sequences of photoactivatable peptides in comparison with the unmodified peptides.

FIG. 2: Competitive binding assay of h/rCRF, rUcn, and their different photoactivatable analogs. Binding curves were normalized by total binding in absence of competitor [B₀]. Data points represent pooled data from three independent experiments.

FIG. 3: Competitive binding assay of h/rCRF⁶⁻³³, rUcn⁵⁻³², and their photoactivatable pBB-analogs. Binding curves were normalized by total binding in absence of competitor [B₀]. Data points represent pooled data from three independent experiments.

FIG. 4: Western blot analysis of rCRFBP and its photoadduct. The dependency of the photoadduct yield on the irradiation time is shown for irradiation without (A) and with (B) filter screen. Panel C represents the densitometric evaluation of immunoblot B.

FIG. 5: Western blot analysis of rCRFBP and its photoadduct. The influence of different competitors on the formation of the photoadduct is shown. RUcn and oCRF were used in a ten-fold excess over pBB-rUcn.

FIG. 6: Western blot analysis of rCRFBP and its photoadduct. Different photoprobes were compared for their photoadduct yields.

FIG. 7: SDS PAGE analysis of the nickel-affinity purified photoreaction products. Samples before (Lane 1) and after (Lane 2) incubation with the affinity matrix are shown in comparison with the eluted fraction (Lane 3). The protein detection was performed by silver staining.

FIG. 8: HPLC-MS of the affinity purified photoreaction products on a C4 column. A linear gradient of 0.4% CH3CN per min was applied. The concentration dependent UV signal enabled the estimation of the photoadduct yield.

FIG. 9: Mass spectrometric peptide mapping of the photoreaction products. The total ion current (TIC) chromatograms represent the HPLC-MS analysis of tryptic fragments derived from rCRFBP (upper) and its photoadduct (lower). Assignment of the fragments was carried out on the basis of the peptide masses. The arrows indicate significant changes in the signal pattern.

FIG. 10: Mass spectrometric peptide mapping of the photoreaction products. The chromatographic profiles (upper: rCRFBP, lower: photoadduct) were created from the analyses shown in FIG. 9 by the application of a selective mass for each peptide to the chromatographic display. The masses of the peptides rCRFBP(97-111) and rCRFBP(100-111) as well as the masses for the corresponding photolabeled products rCRFBP(97-111) and rCRFBP(100-111) (shaded) were applied to both proteolytic digests. The peptides rCRFBP(89-96) and rCRFBP(114-124) flanking the photoreaction site were also displayed as an internal reference. The additional signal for pBB-rUcn(35-40) was due to a mass overlap.

FIG. 11: Electrospray mass spectra of the photolabeled peptides rCRFBP(97-113) (upper) and rCRFBP(100-113) (lower). The spectra were obtained from the HPLC-MS analysis shown in FIG. 9 in the lower panel. In the mass spectrum of rCRFBP(100-113), the co-eluting peptide rCRFBP(234-251) was detected.

FIG. 12: Detection of the photoadducts:

12a: Immunodetection of the photoadducts formed by [Bp⁶]h/rCRF⁶⁻³³ (lanes 1 to 6), [Bp³²]h/rCRF⁶⁻³³ (lanes 7 and 8), and [Bp^{6,32}]h/rCRF⁶⁻³³³ (lanes 9 and 10) and rCRFBP. The absence (−) or presence (+) of photoprobe is indicated. h/rCRF⁶⁻³³ or oCRF (1 μM) were added to the photoprobes (100 nM) and rCRFBP as indicated. The antibodies anti-rCRFBP were used for detection.

12b: Silver-stained SDS gel showing unlabeled rCRFBP and photoadducts both purified by Ni-chelate chromatography. All lanes show the products of photoaffinity-labeling with three different photoprobes. Lane 1: [Bp⁶]h/rCRF⁶⁻³³; lane 2: [Bp³²]h/rCRF⁶⁻³³; lane 3: [Bp^{6,32}]h/rCRF⁶⁻³³.

12c: Immunodetection of the photoadduct formed by [Bp⁶]h/rCRF⁶⁻³³ and rCRFBP. Aliquots of the same sample were analyzed at different time points of irradiation. The bar graphs represent the densitometric evaluation of the intensities of the rCRFBP bands (white) and of the photoadduct bands (black). The photoadduct yields expressed in percentage are included. Prolongation of the irradiation time to 60 min resulted in photodestruction of the proteins as indicated by decreasing signal intensities. The antibodies anti-rCRFBP were used for detection.

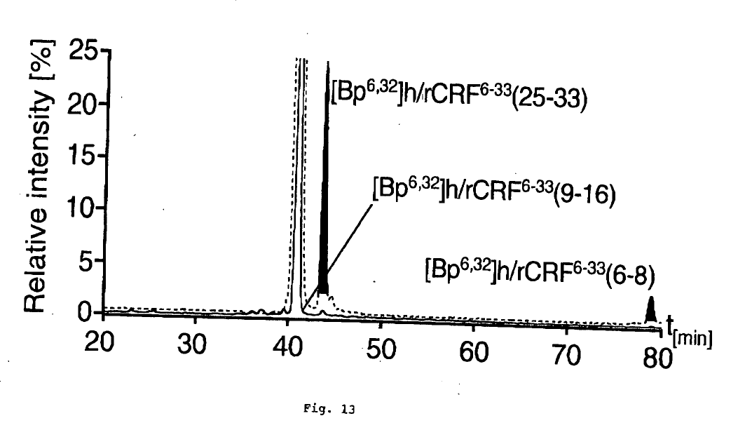

FIG. 13: Peptide mapping of the photoadduct by HPLC-MS

Multiple ion chromatograms (MICs) were extracted from full scan HPLC-MS recordings. The solid trace represents the MIC of the digested photoadduct of rCRFBP and [Bp^{6,32}]h/rCRF⁶⁻³³ on the basis of the following masses: 540.6=[M+H]⁺ of [Bp^{6,32}]h/rCRF⁶⁻³³ (6-8); 507.6=[M+2H]²⁺ of [Bp^{6,32}]h/rCRF⁶⁻³³(9-16); 1127.2=[M+H]⁺ of [Bp^{6,32}]h/rCRF⁶⁻³³(25-33). The superimposed dashed trace represents the MIC of the digested bifunctional photoprobe [Bp^{6,32}]h/rCRF⁶⁻³³ on the basis of the same set of masses. The signals corresponding to the photophore-containing fragments of [Bp^{6,32}]h/rCRF⁶⁻³³ are shaded in grey. The mass-selective chromatograms were scaled to give the same signal height for the internal proteolytic fragment [Bp^{6,32}]h/rCRF⁶⁻³³(9-16) present in both digests.

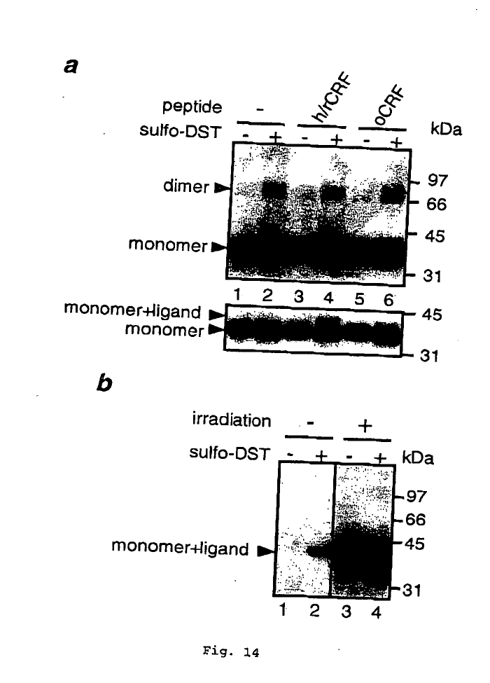

FIG. 14: Subunit structure of rCRFBP

14a: Immunodetection of the cross-linked rCRFBP forms using the antibodies anti-rCRFBP. rCRFBP was incubated without ligand (lanes 1 and 2) or with 50 nM h/rCRF (lanes 3 and 4) or with 50 nM oCRF (lanes 5 and 6) and then cross-linked with sulfo-DST (1 mM). The absence (−) or presence (+) of sulfo-DST is indicated. The bottom part of the figure shows a part of the immunoblot after shortening the exposure time of the same blotting membrane to the X-ray film from 2 min to 10 sec.

14b Immunodetection of the cross-linked rCRFBP forms using the antibodies anti-h/rCRF. rCRFBP was incubated with 50 nM [Bp¹]h/rCRF¹⁻⁴¹ without (lanes 1 and 2) or with subsequent irradiation (lanes 3 and 4), and then cross-linked with sulfo-DST (1 mM). The absence (−) or presence (+) of sulfo-DST is indicated.

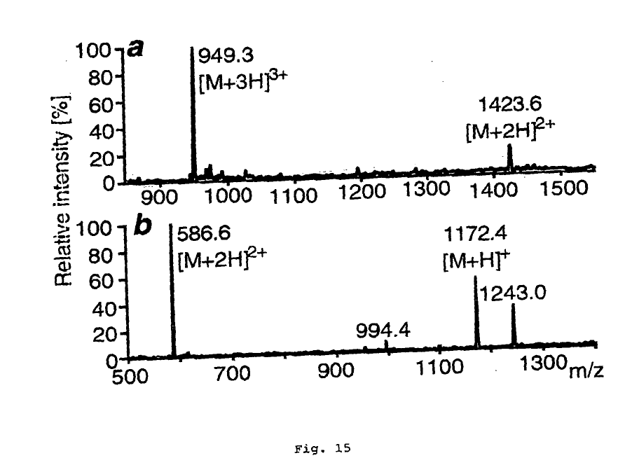

FIG. 15: Mass spectra of the photoadducts

15a: Mass spectrum of the photoadduct fragment rCRFBP(12-26)x[Bp^{6,32}]h/rCRF⁶⁻³³(25-33).

15b: Mass spectrum of the photoadduct fragment rCRFBP(34-38)x[Bp^{6,32}]h/rCRF⁶⁻³³(6-8). The additional signals at m/z=994.4 ([M+5H]⁵⁺) and 1243.0 ([M+4H]⁴⁺) were assigned to the fragment rCRFBP(126-170) co-eluting with the photoadduct fragment.

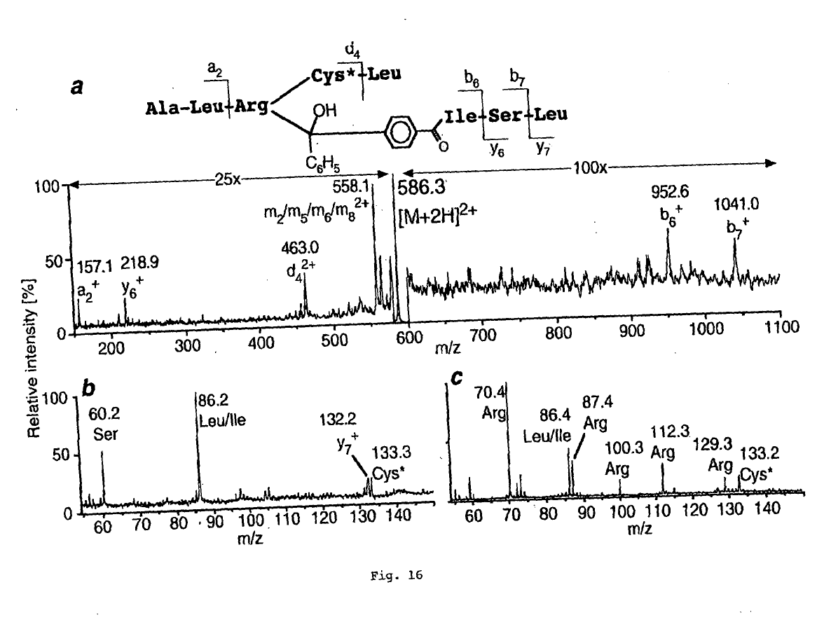

FIG. 16: Analysis of photoadduct fragments by tandem mass spectrometry

16a: High-energy CID mass spectrum of [M+2H]²⁺ of rCRFBP(34-38)x[Bp^{6,32}]h/rCRF⁶⁻³³ (6-8). The derived structure is shown above the spectrum. Cys is marked by an asterisk to indicate S-carboxamidomethylation. Note that the side chain of Cys* is lost with the fragment ion d₄ and thereby excluded as the site of photoincorporation.

16b: Low mass ion region of the high-energy CID mass spectrum shown in FIG. 5a.

16c: Low mass ion region of the high-energy CID mass spectrum of [M+H]⁺ of the synthetic peptide Ala-Leu-Arg-Cys*-Leu representing unlabeled rCRFBP(34-38).

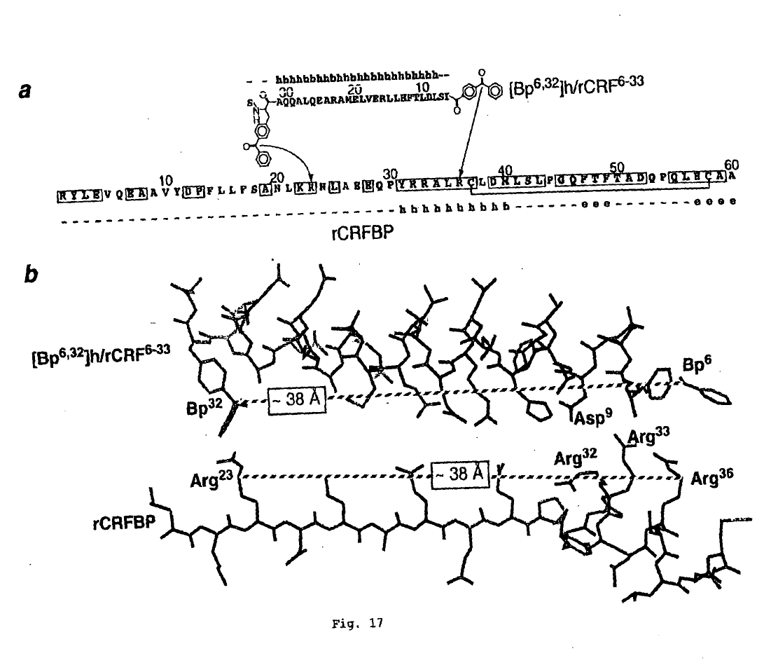

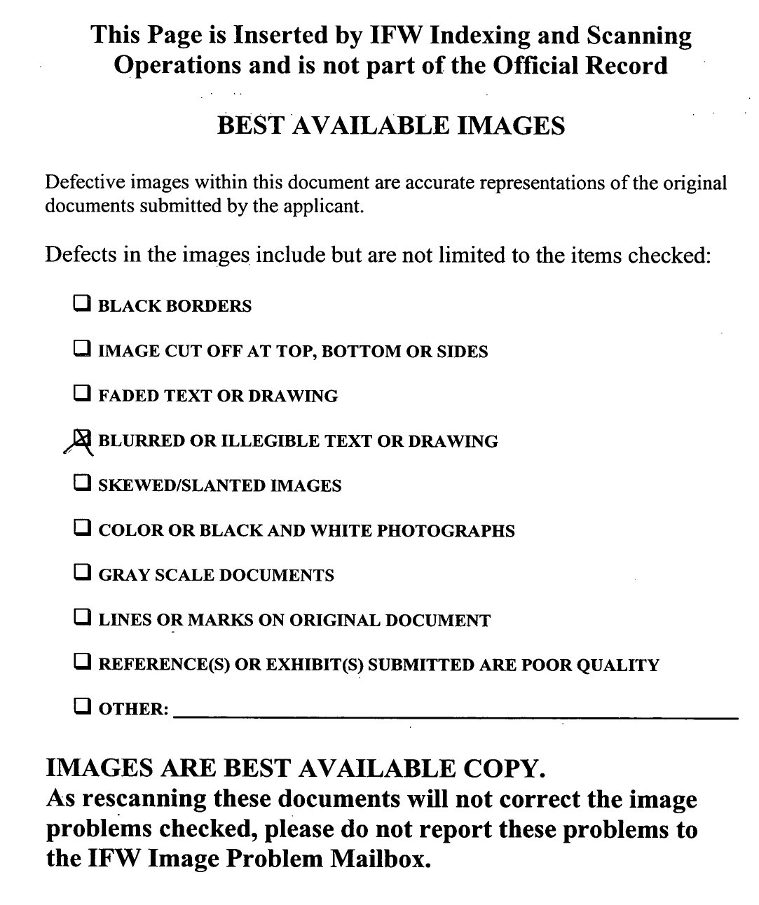

FIG. 17: Proposed interaction of the bifunctional photoprobe with rCRFBP

17a: Schematic representation of the photoaffinity-labeling results. Only the N-terminal 60 of totally 299 amino acid residues of rCRFBP are shown. The amino acid sequence of the ligand including the chemical structures of the photophores is depicted from the C- to the N-terminus to display the anti-parallel alignment. The C-terminal photophore-containing fragment [Bp^{6,32}]h/rCRF⁶⁻³³(25-33) and its site of labeling, rCRFBP(12-26), are shown in green. The N-terminal photophore-containing fragment [Bp^{6,32}]h/rCRF⁶⁻³³(6-8) and its site of labeling, rCRFBP(34-38), are shown in red. The arrows indicate the photolabeled amino acids, Arg²³ and Arg³⁶. The residues conserved between all mammalian CRFBP sequences known to date are boxed. The disulfide bond between Cys³⁷ and Cys⁵⁸ is indicated by a bracket. The secondary structure predicted by the Jnet algorithm is shown above the ligand sequence and below the rCRFBP sequence, respectively. h, helix; e, extended (sheet); −, other (loop).

17b: Wire frame model of [Bp^{6,32}]h/rCRF⁶⁻³³ interacting with the stretch of amino acids 20-40 of rCRFBP. The proposed α-helices were constructed by applying the torsion angles Φ=−57°, ψ=47°, and ω=180°. The extended stretch of amino acids 20-30 of rCRFBP was constructed by applying the torsion angles for a planar polypeptide chain (Φ=−180°, ψ=180°, and ω=180°). In the shown conformations, the distances between Bp⁶ and Bp³² of [Bp^{6,32}]h/rCRF⁶⁻³³, and Arg²³ and Arg³⁶ of rCRFBP were both approximately 38 Å. Asp⁹ of [Bp^{6,32}]h/rCRF⁶⁻³³ may bind to either Arg³² or Arg³³ of rCRFBP by an electrostatic interaction.

The Examples Illustrate the Invention

### EXAMPLE 1

### Peptide Synthesis

All peptides were synthesized using an ABI 433A peptide synthesizer (PE Biosystems, Weiterstadt, Germany). Stepwise coupling of amino acids to the respective resin followed the fluorenylmethoxycarbonyl (Fmoc) chemistry option with HBTU activation. TentaGel S Ram resin (Rapp, Tübingen, Germany) was used for C-terminally amidated peptides whereas HMP resin (PE Biosystems, Weiterstadt, Germany) was used for the truncated CRF analogs h/rCRF⁶⁻³³ and rUcn⁵⁻³² which incorporate a C-terminal carboxyl group. pBF¹¹-rUcn was synthesized by using the commercially available Fmoc-para-benzoyl-Phe (Bachem, Heidelberg, Germany) instead of Phe¹¹. For N-terminal modification, para-benzoylbenzoic acid N-hydroxysuccinimide ester was coupled to the deprotected α-amino group of the resin-linked peptide. The reaction was carried out overnight at room temperature in dimethylformamide with a five-fold excess of the activated ester in the presence of a two-fold excess of diisopropylethylamine. The peptides were cleaved from the resin under standard conditions. The crude peptides were purified by reversed-phase HPLC (RP-HPLC) on Vydac C4 and C18 columns (250×22 mm, 10 μm particles, 300 Å pore size; Vydac, Hesperia, Calif.) and characterized with HPLC-mass spectrometry (HPLC-MS) and amino acid analysis, respectively.

### EXAMPLE 2

### HPLC-Mass Spectrometry and Amino Acid Analysis

HPLC-MS was performed by using an HPLC system composed of an ABI 140A syringe pump, an ABI 759A single wavelength UV detector (PE Biosystems, Weiterstadt, Germany), and an IC CAP 100 gradient splitter (LC Packings, Amsterdam, NL). Separations were carried out on columns of 0.3 mm ID and 15 cm length. The columns were packed with Vydac C4 and C18 material of 5 μm particle and 300 Å pore size (LC Packings, Amsterdam, NL). Gradients of water and acetonitrile containing 0.05 to 0.07% trifluoroacetic acid were applied for elution. The HPLC eluent passed through the UV detector was directly infused into the elelctrospray interface of a AutoSpec-T mass spectrometer (Micromass, Manchester, UK). UV and mass spectral data were recorded by the OPUS 3.5 data system.

Amino acid analysis was performed after hydrolysis of peptides (6 M HCl, 3 h, 150° C.) with a Beckman HPLC Analyzer System 6300 (Beckman Coulter, Fullerton, Calif.).

### EXAMPLE 3

### Production of rCRFBP in HEK 293 Cells

For eucaryotic expression, a cDNA fragment coding for the 322 amino acid rCRFBP precursor protein was amplified by PCR introducing a sequence coding for a His₆-sequence at the 3′-end. The construct was cloned into the eucaryotic expression vector pcDNA3 (Invitrogen, San Diego, Calif.) utilizing the restriction enzymes KpnI and EcoRI.

HEK 293 cells were maintained as described (15) and transfected using the calcium phosphate-DNA co-precipitation method (16). Two days after transfection of HEK 293 cells with 10 μg plasmid DNA, medium was added containing the selective antibiotic geneticin 418 sulfate (Gibco BRL, Eggenstein, Germany) at a final concentration of 625 μg/ml. Stably transfected HEK 293 cells were grown in regular FCS-supplemented medium until approximately 80% confluent. They were then switched to serum free medium Nephros LP (BioWhittaker, Walkersville, Md.) supplemented with selective antibiotic as described above and 2 mM L-glutamine. The cells were maintained in Nephros LP without further subculturing, and medium was collected at different time points. The medium was tested for the presence of rCRFBP by SDS PAGE combined with Western blotting and immunodetection.

### EXAMPLE 4

### SDS PAGE, Western Blotting, and Immunodetection

SDS PAGE and Western blot analysis were performed as previously described (17, 18). Electrophoreses were run on 9% polyacrylamide gels. For immunodetection of rCRFBP and the respective photoadducts, a polyclonal anti-rCRFBP antibody produced in rabbits was used. The employed antigen corresponding to rCRFBP lacking the N-terminal 24 amino acid signal sequence was expressed in E. coli according to Sydow et al. (19). The cDNA fragment coding for rCRFBP was amplified by PCR and cloned into the procaryotic expression vector pQE70 (Qiagen, Hilden, Germany) utilizing the restriction enzymes SphI and BgIII. Anti-rCRFBP was used as primary antibody in Western blot analysis at a final concentration of 0.5 μg/ml. The secondary antibody was conjugated to alkaline phosphatase and protein detection by chemoluminescence was applied (19). For protein detection by silver staining, a standard protocol according to Merril et al. (20) was used.

### EXAMPLE 5

### Radioligand Binding Assay and Data Analysis

The peptides were tested for their affinity to rCRFBP on the basis of a charcoal precipitation assay utilizing tritiated Ucn as radioligand (21). The binding assay consisted of 0.4 nM ³H-rUcn (80 Ci/mmol; Amersham Pharmacia Biotech, Uppsala, Sweden) and 1 μl medium from HEK 293 cells expressing the rCRFBP in a total volume of 300 μl PBS, pH 7.5, containing 0.02% (w/v) nonionic detergent NP-40. Siliconized microreaction vials (Sigma, Deisenhofen, Germany) were used to prevent binding of peptides to plastic surfaces. Competition binding experiments were performed by employing 0 to maximally 3 μM unlabeled competitor. The applied concentration range depended on the expected order of magnitude of ligand affinity. After 2 hours of incubation at room temperature, unbound ligand was precipitated by the addition of 100 μl precipitation buffer (binding buffer+10% w/v activated charcoal+2% BSA). The suspension was mixed vigorously and the adsorption was allowed to proceed for 2 min at room temperature. After centrifugation for 2 min at 12000×g, 200 μl supernatant was removed, mixed with 2 ml Ecolume liquid scintillation cocktail (ICN Biomedicals, Irvine, Calif.), and counted for 3 min in a Wallac 1209 Rackbeta liquid scintillation counter (Wallac, Turku, Finland). All assays were performed in duplicate and repeated at least three times. Binding data were analyzed using the Prism computer program (GraphPad Software, San Diego, Calif.). The K_{D} value for rUcn obtained from homologous competition experiments was used for the calculation of K_{I} values according to the Cheng-Prusoff equation (22).

### EXAMPLE 6

### Photoaffinity Labeling of rCRFBP

rCRFBP (approximate final concentration 10 nM) was incubated with 100 nM of the photoactivatable peptide according to the conditions of the radioligand binding assay. Medium from stably transfected HEK 293 cells was directly used as source for rCRFBP. Irradiation of the benzophenone photoprobes was carried out at 0° C. with a self-constructed photoreaction device incorporating a 400 W halogen metal vapor lamp (Ultratech, Osram, Germany). This lamp provides a wide spectrum of emission at wavelenghts above 250 nm with a maximum at 370 nm which is consistent with the activation wavelenght (350-360 nm) for benzophenone derivatives (13). Protein-damaging wavelenghts below 300 nm were avoided by the use of a filter screen consisting of the optical glass type B 270 (Schoft, Mainz, Germany). The aryldiazirine photoprobe was irradiated as described (14) using a commercially available stratalinker device.

### EXAMPLE 7

### Mass Spectrometric Peptide Mapping of the Photoreaction Products

The photoreaction products were purified by nickel-affinity chromatography under denaturing conditions as batch procedure using the buffer systems suggested by the supplier (Qiagen, Hilden, Germany). The purified proteins were derivatized by means of cysteine alkylation under reducing conditions. The reaction was carried out as described (23) with the modification that dithiothreitol (DTT) and 2-iodoacetamide were used instead of mercaptoethanol and 2-iodoacetic acid. After S-carboxamidomethylation, the photoadduct was separated from unlabeled rCRFBP by RP-HPLC and both proteins were subjected to proteolysis utilizing TPCK-trypsin (Sigma, Deisenhofen, Germany). Tryptic digests were carried out in HEPES buffer (pH 7.0) containing 2 M urea, 5% acetonitrile, and 5 mM CaCl₂ with an enzyme substrate ratio of 1:50 (w/w). The digests were incubated for 2 h at 37° C. and analyzed on HPLC-MS.

### EXAMPLE 8

### The Binding Protein of Corticotropin-Releasing Factor: Ligand Binding Site and Subunit Structure

Introduction

Corticotropin-releasing factor (30, 31) (CRF), the key mediator of the mammalian responses to stress stimuli, has also been recognized as an important neuromodulator of brain functions such as learning and anxiety (32). The central actions of CRF are mediated through at least two different subtypes of CRF receptors (CRFRs), CRFR1 and CRFR2 (33), and are modulated by a 37 kDa CRF binding protein (34) (CRFBP), which is localized in several distinct brain regions including the cerebral cortex and the hippocampus (35). Consistent with its proposed role to reduce the ligand's availability for CRFR-mediated actions (35, 36), it has been shown that 40 to 60% of the human brain CRF is bound by CRFBP (37). Thus, the binding protein can be considered as a physiologically relevant reservoir of endogenous CRF.

CRFR1 and 2 mediate opposite effects on learning and anxiety. Learning is enhanced through hippocampal CRFR1, whereas it is impaired through septal CRFR2 (38). Anxiety-like behavior is increased by activation of CRFR1 and predominantly decreased by activation of CRFR2 as indicated by CRFR1 and CRFR2 gene deletions (39-42). In this complex situation, selective activation of CRFR-dependent brain functions could be achieved on the basis of the distinct distribution of CRFBP in the brain. Thus, the release of endogenous ligand from hippocampal CRFBP could increase memory consolidation under physiologic and pathophysiologic conditions (37, 38) without producing anxiety-like effects through CRFR2 of the lateral septum void of CRFBP. Displacement of CRF from its binding protein may be achieved by CRFBP-selective peptides (CRFBP-inhibitors) such as human/rat (h/r) CRF⁶⁻³³ (37, 43), a synthetic fragment of h/rCRF. A detailed knowledge on the ligand binding site of CRFBP, whose three-dimensional structure has not been resolved so far, may facilitate the design of new peptidic and non-peptidic CRFBP-inhibitors.

We identified using photoaffinity-labeling the ligand binding site of the rat CRFBP (rCRFBP). New photoreactive analogs of h/rCRF⁶⁻³³ were employed in combination with different mass spectrometric techniques to directly determine contact sites between residues of CRF and its binding protein. In view of the finding that human CRFBP dimerizes after association with ligand (44), the subunit structure of rCRFBP was investigated with mono- and bifunctional photoprobes as well as by chemical cross-linking.

Methods

Peptide Synthesis

All peptides were synthesized by standard solid phase synthesis using Fmoc chemistry as described (45). Fmoc-para-benzoyl-Phe (Bachem) was used to introduce the benzophenone photophore into the polypeptides. For N-terminal modification, para-benzoylbenzoic acid N-hydroxysuccinimide ester (Molecular Probes) was coupled to the de-protected α-amino group of the resin-linked peptide. The reaction was carried out overnight at room temperature in dimethyl formamide with a five-fold molar excess of the activated ester.

Production of rCRFBP and Binding Assay

Recombinant rCRFBP containing a C-terminal His-tag was produced in human embryonic kidney (HEK) 293 cells under serum-free conditions (45). Binding of peptides to rCRFBP was determined in PBS/0.02% NP-40 using a scintillation proximity assay with [¹²⁵I-Tyr⁰]h/rCRF as radioligand (46).

Generation and Detection of the Photoadducts

For photoaffinity-labeling, medium containing rCRFBP was incubated with the photoprobe (100 nM) under the conditions of the binding assay. Unless otherwise stated, activation was carried out for 30 min at 0° C. with an Ultratech 400 W halogen metal vapor lamp (Osram). Protein-damaging wavelenghts below 300 nm were filtered with a B270 glass screen (Schott). Unlabeled rCRFBP and the photoadduct were co-purified by adsorption to a nickel-chelate matrix under denaturing conditions. The eluted fraction was analyzed by SDS PAGE (45) (9% polyacrylamide gels) and by reversed-phase HPLC coupled on-line to mass spectrometry (HPLC-MS) on a Vydac C4 column (0.3×150 mm; LC Packings). Western blot analysis with chemoluminescence detection using the polyclonal antibodies anti-rCRFFP (45) or anti-h/rCRF (Sigma) was carried out as described (45).

Chemical Cross-Linking

rCRFBP was cross-linked by incubation without or with ligand (50 nM) under the conditions of the binding assay and subsequent treatment with 1 mM sulfo-DST (Pierce) for 1 h at 20° C.

Peptide Mapping of the Photoadducts by HPLC-MS

S-carboxamidomethylation of Cys residues by iodoacetamide and peptide mapping of rCRFBP monitored by mass spectrometric analysis was performed as described recently (45). The photoadduct was separated from unlabeled rCRFBP by HPLC on a Vydac C4 column (1.0×150 mm; LC Packings). Both protein species were first digested with endoprotease AspN and subsequently with TPCK-trypsin. The resulting peptide mixtures were analyzed by HPLC-MS either on a PepMap C18 or a Vydac LowTFA C4 column (0.3×150 mm; LC Packings). Gradients formed by 0.07% formic acid and 0.05% formic acid containing 80% acetonitrile were applied. These columns were also used to fractionate enzymatic digests for off-line tandem mass spectrometry.

Tandem Mass Spectrometry

The high energy collision-induced dissociation (CID) mass spectra were recorded on an Autospec-T four sector tandem mass spectrometer (Micromass) equipped with a nanoelectrospray (NanoES) ion source and a multichannel array detector. The NanoES glass capillaries (Protana) were filled with 1 μl of the peptide samples. Argon was used as collision gas with an adjusted pressure to provide an attentuation of the ion beam by 70% (singly charged precursor ions) or by 90% (multiply charged precursor ions). The ion-accelerating voltage was 4 kV; the gas cell was operated at 2 kV above ground potential. The fragment ions were annotated as proposed by Tuinman and Pettit (47).

Prediction of Secondary Structure

Secondary structure predictions were performed using the Jnet prediction method (48) which is available in the Jpred server from http://barton.ebi.ac.uk. A multiple sequence alignment of the four known mammalian CRFBP amino acid sequences from different species (rat, mouse, human, sheep) was submitted to the server.

Results

Design of the Photoprobes

The photoprobes were designed on the basis of the amino acid sequence of h/rCRF⁶⁻³³ representing the minimal sequence required for high affinity binding to CRFBP (43). The benzophenone (Bp) photophore was introduced into h/rCRF⁶⁻³³ either at the N-terminus by modification of the α-amino group to generate [Bp⁶]h/rCRF⁶⁻³³, or at the C-terminus by replacement of the bulky His residue in position 32 by a para-benzoyl-Phe residue to generate [Bp³²]h/rCRF⁶⁻³³. By combining these modifications, the bifunctional photoprobe [Bp^{6,32}]h/rCRF⁶⁻³³ (see FIG. 17a for structure) was obtained to be used as a specific cross-linker covalently connecting two remote contact points between h/rCRF⁶⁻³³ and CRFBP. The affinities of the monofunctional photoprobes [Bp⁶]h/rCRF⁶⁻³³ (IC₅₀=3.2 nM; 95% confidence interval (CI): 2.3-4.0 nM) and [Bp³²]h/rCRF⁶⁻³³ (IC₅₀=2.1 nM; 95% CI: 0.7-3.5 nM) were not significantly different from the affinity of h/rCRF⁶⁻³³ to rCRFBP (IC₅₀=1.9 nM; 95% CI: 1.3-2.5 nM). The affinity of the bifunctional photoprobe [Bp^{6,32}]h/rCRF⁶⁻³³ (IC₅₀=14 nM; 95% CI: 11-18 nM) was decreased by a factor of seven compared to that of h/rCRF⁶⁻³³, but despite this reduction neither the specificity nor the yield of photoadduct formation was detectably affected.

Detection of the Photoadducts

By Western blot analysis using specific polyclonal antibodies directed against rCRFBP (45) (anti-rCRFBP), 41 kDa species corresponding to the photoadducts of rCRFBP were detected (FIG. 12a). As representatively shown for [Bp⁶]h/rCRF⁶⁻²³ (FIG. 12a), the formation of all photoadducts was completely suppressed in the presence of an excess of h/rCRF⁶⁻³³, but not in the presence of a low affinity ligand such as ovine CRF, demonstrating the specificity of the photoprobe binding.

rCRFBP and its respective photoadduct were co-purified by nickel-chelate chromatography employing the C-terminal His-tag fused to recombinant rCRFBP (45). Analysis of the obtained fractions with SDS PAGE followed by silver staining (FIG. 12b), or with HPLC-MS (data not shown), revealed photoadduct yields of approximately 50% which were similar to those observed by immunodetection (FIG. 12a). This markedly high yield of photoincorporation may have resulted from the reported slow dissociation of ligands from CRFBP (20) in combination with the reversible activation of benzophenone photophores via excitation-relaxation cycles (50).

It has earlier been proposed that CRF induces dimerization of CRFBP (44). Thus, it was conceivable that the ligand was bound to one or both subunits of this putative CRFBP complex. These possibilities were investigated with the bifunctional photoprobe [Bp^{6,32}]h/rCRF⁶⁻³³. It was demonstrated that the sites of photoincorporation of the monofunctional photoprobes [Bp⁶]h/rCRF⁶⁻³³ and [Bp³²]h/rCRF⁶⁻³³ resided in the same subunit of the putative rCRFBP dimer. By SDS PAGE, only a 41 kDa species corresponding to the photoadduct formed by rCRFBP and [Bp^{6,32}]h/rCRF⁶⁻³³ was detected (FIG. 12a, 12b). The absence of a dimer species indicated that the bifunctional photoprobe did not interlink different subunits. To verify that the photoprobe's benzophenone groups both had reacted with rCRFBP, proteolytic digests of the photoadduct formed by rCRFBP and [Bp^{6,32}]h/rCRF⁶⁻³³, and of the bifunctional photoprobe alone were analyzed by the extraction of multiple ion chromatograms from full scan HPLC-MS recordings (51) (FIG. 13). Thereby, the photophore-containing ligand fragments were detected in the digest of [Bp^{6,32}]h/rCRF⁶⁻³³, but not in the digest of the photoadduct. By this finding, it was demonstrated that the N-terminal as well as the C-terminal benzophenone groups of [Bp^{6,32}]h/rCRF⁶⁻³³ reacted almost quantitatively with rCRFBP.

When the photoadduct formation of rCRFBP and [Bp⁶]h/rCRF⁶⁻³³ was analyzed at different time points of irradiation, yields up to approximately 65% were observed (FIG. 12c). These results were suggestive that the photoadduct formation took place on the level of a CRFBP monomer labeled by one ligand molecule.

Subunit Structure of rCRFBP

The subunit structure of rCRFBP under the conditions of photoaffinity-labeling was further analyzed by chemical cross-linking using sulfo-disuccinimidyl tartrate (sulfo-DST) in the absence or presence of different ligands. In agreement with the observed different affinities to rCRFBP, h/rCRF, but not oCRF was found to be cross-linked to the binding protein as indicated by Western blot analysis using anti-rCRFBP for detection (FIG. 14a). In addition, treatment of rCRFBP with sulfo-DST generated species of 65-80 kDa consistent with the size of dimeric species (FIG. 14a). The appearance of the dimer in the gel as a doublet with an apparent difference in size of approximately 10 kDa was probably not due to glycosylation differences since protein chemical characterization revealed that recombinant rCRFBP carried a single Asn-linked carbohydrate moiety with an average mass of 1.5 kDa (45). The doublet may be rather explained by two distinct protein species formed during cross-inking and displaying different electrophoretic mobilities in SDS PAGE. No aggregates larger than dimers were observed. It was therefore concluded that dimerization was not due to unphysiological high concentrations of rCRFBP. On the basis of Bₘₐₓ values derived from homologous competition binding curves (45), the concentration of rCRFBP in the cross-linking experiment was estimated to be in the range of 1-10 nM in agreement with the plasma levels of CRFBP in humans (52, 53).

Surprisingly, the intensity of the dimer bands did not depend of the presence of h/rCRF or oCRF, and dimerization was even detected in the absence of any added ligand (FIG. 14a). These results were suggestive that the dimer formation took place on the basis of a ligand-independent mechanism. For further analysis of the composition of the dimeric species, chemical cross-linking was carried out in the presence of the photoprobe [Bp¹]h/rCRF¹⁻⁴¹ without and with prior irradiation. The cross-linked species were visualized by immunodetection of the photoprobe using polyclonal antibodies directed against h/rCRF²⁵⁻⁴¹ (anti-h/rCRF). In agreement with the photoprobe's high affinity to rCRFBP (IC₅₀=0.67 nM; 95% CI: 0.40-0.93 nM), a 42 kDa species corresponding to [Bp¹]h/rCRF¹⁻⁴¹ chemically cross-linked to rCRFBP was detected after treatment with sulfo-DST (FIG. 14b; lane 2). When [Bp¹]h/rCRF¹⁻⁴¹ was covalently linked to rCRFBP by irradiation prior to treatment with sulfo-DST, the signal intensity of this 42 kDa band was significantly increased (FIG. 14b; lane 4). However, despite the covalent attachment of the ligand to rCRFBP prior to chemical cross-linking, no signals were detected in the range of 65-80 kDa where a dimeric species would have been expected to appear (FIG. 14b). Thus, it was demonstrated that the ligand was not part of the rCRFBP dimer. It was therefore concluded, that the dimer was not involved in ligand binding.

Identification of the Ligand Binding Site

After S-carboxamidomethylation of the Cys residues with iodoacetamide, the photoadduct was separated from unlabeled rCRFBP by reversed-phase HPLC on the basis of the significantly increased hydrophobic properties of the photoadduct due to covalent attachment of the photoprobe (data not shown). The isolated photoadduct was digested with endoprotease AspN and subsequently with trypsin. By combining these two proteases, relatively small protein fragments (<3 kDa) were obtained which were then analyzed with HPLC-MS and thus characterized on the basis of their molecular masses. Unlabeled rCRFBP obtained from the same labeling experiment was digested similarly and used as control. Peptides with observed molecular masses that were incompatible with the calculated rCRFBP and photoprobe fragments, respectively, were considered as photoadduct-specific fragments and further analyzed.

By HPLC-MS analysis of the digested photoadduct formed by rCRFBP and the bifunctional photoprobe [Bp^{6,32}]h/rCRF⁶⁻³³, two characteristic molecular masses were identified and unambiguously assigned to proteolytic photoadduct fragments. A mass of M_{obs}=2845.1 (FIG. 15a) was assigned to rCRFBP(12-26) labeled by [Bp^{6,32}]h/rCRF⁶⁻³³(25-33) (M_{calc}=2845.24; ΔM=50 ppm). A mass of M_{obs}=1171.3 (FIG. 15b) was assigned to rCRFBP(34-38) photolabeled by [Bp^{6,32}]h/rCRF⁶⁻³³ (6-8) (M_{calc}=1171.43; ΔM=110 ppm). Thus, rCRFBP(12-26) and rCRFBP(34-38) were identified as the binding sites of the C- and the N-terminal part of the bifunctional photoprobe, respectively (FIG. 17a). In agreement with this finding, the monofunctional photoprobes [Bp³²]h/rCRF⁶⁻³³ and [Bp⁶]h/rCRF⁶⁻³³ labeled rCRFBP(12-26) and rCRFBP(34-38), respectively (data not shown). The tryptic cleavage sites Lys²², Arg²³, and Arg³⁶, that were found to be cleaved in the control digest of unlabeled rCRFBP, were blocked as a result of the photolabeling indicating that the modification occurred directly at or in close proximity to the respective amino acid residue.

For further characterization, proteolytic digests of the photoadducts were fractionated by HPLC. Fractions containing the labeled peptides were analyzed by tandem mass spectrometry. The sequence of the photoadduct fragment rCRFBP(34-38)x[Bp^{6,32}]h/rCRF⁶⁻³³ (6-8) was deduced from its high-energy CID mass spectrum (FIG. 16a). Thereby, a covalent linkage of rCRFBP at Arg³⁶ to [Bp^{6,32}]h/rCRF⁶⁻³³ (6-8) was demonstrated. The same result was obtained for the photoadduct fragment rCRFBP(34-38)x[Bp⁶]h/rCRF⁶⁻³³ (6-8) formed by the monofunctional photoprobe [Bp⁶]h/rCRF⁶⁻³³ (data not shown). Consistent with the modification of the side chain of Arg³⁶, the low mass fragment ions at m/z=70, 87, 100, 112, and 129 (54) indicative for the presence of Arg were absent in the spectrum of the photoadduct fragment (FIG. 16b). In contrast, all low mass ion signals were present in the spectrum of the corresponding synthetic peptide rCRFBP(34-38) containing Arg³⁶ unmodified (FIG. 16c).

The high-energy CID mass spectrum of the photoadduct fragment rCRFBP(12-26)x[Bp^{6,32}]h/rCRF⁶⁻³³(25-33) did not reveal any fragment ion signals indicative for the cleavage of the peptide backbone of rCRFBP(12-26). Therefore, the CID mass spectra of the photoadduct fragment and of synthetic rCRFBP(12-26) were compared with respect to their low mass ion regions as described above (data not shown). The lack of the low mass ion signals at m/z=100, 112, and 129 indicated the labeling of the side chain of Arg²³ by [Bp^{6,32}]h/rCRF⁶⁻³³ (25-33). In agreement with the presence of Pro¹³ and Asn^{20,24} in rCRFBP(12-26), the low mass ion signals at m/z=70 and 87 (25) were not absent, but their intensities were significantly decreased. The same observations held for the photoadduct fragment rCRFBP(12-26)x[Bp^{6,32}]h/rCRF⁶⁻³³(25-33) obtained with the monofunctional photoprobe [Bp³²]h/rCRF⁶⁻³³ (data not shown).

Within the amino acid side chains, methyl or methylene groups adjacent to heteroatoms are known to be particulary reactive sites for photoincorporation of benzophenone groups (50). In agreement with this preference, labeling of the Met side chain was found in the majority of photoaffinity-labeling studies employing benzophenone-derivatized peptides (55-57). However, the labeling of the Arg side chain observed here was also consistent with the reaction preference of benzophenones. Despite the uncertainty resulting from this preference, the photoaffinity-labeling of rCRFBP was considered to be highly regio-specific as indicated by the finding that only one arginine, Arg³⁶, of three Arg residues within the stretch of amino acids 32-36 was labeled (FIG. 17a).

Discussion

In view of the composition of the CRFBP binding complex, we concluded from our data that one molecule of h/rCRF was bound to a rCRFBP monomer. This conclusion contrasts with the assumption of the ligand-induced dimerization of human CRFBP as was proposed on the basis of gel filtration data (44). It remains to be established, whether the different mode of ligand-interaction of human CRFBP is related to its production outside of the brain and pituitary as is limited to humans (34, 36).

On the basis of the results of photoaffinity-labeling, we propose an anti-parallel alignment of [Bp^{6,32}]h/rCRF⁶⁻³³ with the N-terminal domain of rCRFBP during binding (FIG. 17). Our data indicated that in the bound state, Ile⁶ and para-benzoyl-Phe³² of [Bp^{6,32}]h/rCRF⁶⁻³³ were in close proximity to Arg³⁶ and Arg²³ of rCRFBP, respectively. Thus, it was probable that the 28 residue photoprobe spanned over the 14 residue polypeptide chain of rCRFBP. By using CD and NMR spectroscopic methods, evidence has been provided that h/rCRF forms a defined amphiphilic α-helix in the central part of the molecule (58, 59). Therefore, a similar helical structure was assumed for [Bp^{6,32}]h/rCRF⁶⁻³³. To fit into the proposed alignment, the interacting polypeptide chain of rCRFBP needed to adopt a more stretched conformation, which was compatible with the secondary structure predicted by the Jnet prediction method (48) (FIG. 17).

Recently, we have demonstrated that Ala²² of h/rCRF plays a crucial role in high affinity binding to rCRFBP (46). Since this amino acid is part of the hydrophobic patch of the amphiphilic α-helix of h/rCRF, we proposed an involvement of the hydrophobic patch in binding to rCRFBP (46). In the present study, we identified a relatively hydrophilic stretch of rCRFBP as contact site between h/rCRF and its binding protein (FIG. 17). In agreement with the model developed here, it is suggested that the hydrophilic patch of h/rCRF was in close contact with the stretch of residues 23-36 of rCRFBP (FIG. 17), whereas the hydrophobic patch of h/rCRF remained available for interaction with a different—presumably more hydrophobic—part of rCRFBP. Interestingly, the stretch of amino acids 3140 of rCRFBP was the unique part of the entire protein predicted to form an α-helical secondary structure. Since residues 31-40 are highly conserved between the mammalian CRFBP sequences known to date (60), it was likely that a helix-helix interaction may be involved in ligand binding of CRFBP. In view of this assumption, an ionic interaction of Asp⁹ of [Bp^{6,32}]h/rCRF⁶⁻³³ with Arg³² or Arg³³ of rCRFBP may have contributed to the stability of the complex. Asp⁹ is completely conserved by all high-affinity ligands of CRFBP, and its absence in the CRF antagonist astressin designed on the basis of the sequence of h/rCRF¹²⁻⁴¹ (32) may be responsible for the low affinity of this peptide compared to the high affinity of the CRF antagonist α-helical CRF⁹⁻⁴¹ (46).

### REFERENCES

1. Spiess J, Rivier J, Rivier C, and Vale W W (1981) Primary structure of corticotropin-releasing factor from ovine hypothalamus. Proceedings of the National Academy of Sciences, United States of America 78:6517-6521.

2. Vale W W, Spiess J, Rivier C, and Rivier J (1981) Characterization of a 41-residue ovine hypothalamic peptide that stimulates secretion of corticotropin and beta-endorphin. Science 213:1394-1397.

3. Vaughan J, Donaldson C, Bittencourt J, Perrin M H, Lewis K, Sutton S, Chan R, Turnbull A V, Lovejoy D, Rivier C, Rivier J, Sawchenko P E, and Vale W W (1995) Urocortin, a mammalian neuropeptide related to fish urotensin I and to corticotropin-releasing factor. Nature 378:287-292.

4. Spiess J, Dautzenberg F M, Sydow S, Hauger R L, Rühmann A, Blank T, and Radulovic J (1998) Molecular properties of the CRF receptor. Trends in Endocrinology & Metabolism 9:140-145.

5. Behan D P, Linton E A, and Lowry P J (1989) Isolation of the human plasma corticotrophin-releasing factor-binding protein. Journal of Endocrinology 122:23-31.

6. Potter E, Behan D P, Fischer W H, Linton E A, Lowry P J, and Vale W W (1991) Cloning and characterization of the cDNAs for human and rat corticotropin releasing factor-binding proteins. Nature 349:423-b 426.

7. Potter E, Behan D P, Linton E A, Lowry P J, Sawchenko P E, and Vale W W (1992) The central distribution of a corticotropin-releasing factor (CRF)-binding protein predicts multiple sites and modes of interaction with CRF. Proceedings of the National Academy of Sciences of the United States of America 89:4192-4196.

8. Karolyi I J, Burrows H L, Ramesh T M, Nakajima M, Lesh J S, Seong E, Camper S A, and Seasholtz A F (1999) Altered anxiety and weight gain in corticotropin-releasing hormone-binding protein-deficient mice. Proceedings of the National Academy of Sciences of the United States of America 96:11595-11600.

9. Behan D P, Grigoriadis D E, Lovenberg T, Chalmers D, Heinrichs S, Liaw C, and De S E B (1996) Neurobiology of corticotropin releasing factor (CRF) receptors and CRF-binding protein: implications for the treatment of CNS disorders. Molecular Psychiatry 1:265-277.

10. Sutton S W, Behan D P, Lahrichi S L, Kaiser R, Corrigan A, Lowry P, Potter E, Perrin M H, Rivier J, and Vale W W (1995) Ligand requirements of the human corticotropin-releasing factor-binding protein. Endocrinology 136:1097-1102.

11. Behan D P, Heinrichs S C, Troncoso J C, Liu X J, Kawas C H, Ling N, and De SEB (1995) Displacement of corticotropin releasing factor from its binding protein as a possible treatment for Alzheimer's disease. Nature 378:284-287.

13. Dorman G, and Prestwich G D (1994) Benzophenone photophores in biochemistry. Biochemistry 33:5661-5673.

14. Rühmann A, Kopke A K, Dautzenberg F M, and Spiess J (1996) Synthesis and characterization of a photoactivatable analog of corticotropin-releasing factor for specific receptor labeling. Proceedings of the National Academy of Sciences of the United States of America 93:10609-10613.

15. Graham F L, Smiley J, Russell W C, and Nairn R (1977) Characteristics of a human cell line transformed by DNA from human Adenovirus type 5. Journal of Gene Virology 36:59-72.

16. Sambrook J, Fritsch E F, and Maniatis T (1989) Molecular cloning: a laboratory manual. Cold Spring Harbor Laboratory Press, Plainview, N.Y., 2^{nd} Edition.

17. Köpke AKE, Bonk I, Sydow S, Menke H, and Spiess J (1993) Characterization of the NR1, NR2A, and NR2C receptor proteins. Protein Science 2:2066-2076.

18. Sydow S, Köpke A K E, Blank T, and Spiess J (1996) Overexpression of a functional NMDA receptor subunit (NMDAR1) in baculovirus-infected Trichoplusiani insect cells. Molecular Brain Research 41:228-240.

19. Sydow S, Radulovic J, Dautzenberg F M, and Spiess J (1997) Structure function relationship of different domains of the rat corticotropin-releasing factor receptor. Molecular Brain Research 52:182-193.

20. Merril C R, Goldman D, Sedman S A, and Ebert M H (1981) Ultrasensitive stain for proteins in polyacrylamide gels shows regional variation in cerebrospinal fluid proteins. Science 211:1437-1438.

21. Ardati A, Gottowik J, Henriot S, Clerc R G, and Kilpatrick G J (1998) Pharmacological characterisation of the recombinant human CRF binding protein using a simple assay. Journal of Neuroscience Methods 80:99-105.

22. Cheng Y, and Prusoff W H (1973) Relationship between the inhibition constant (Kᵢ) and the concentration of inhibitor which causes 50 percent inhibition (I₅₀) of an enzymatic reaction. Biochemical Pharmacology 22:3099-3108.

23. Crestfield A M, Moore S, and Stein W H (1963) The preparation and enzymatic hydrolysis of reduced and S-carboxymethylated proteins. Journal of Biological Chemistry 238:622-627.

24. Behan D P, Cepoi D, Fischer W H, Park M, Sutton S, Lowry P J, and Vale W W (1996) Characterization of a sheep brain corticotropin releasing factor binding protein. Brain Research 732:265-274.

25. Kasheverov I E, Utkin Y N, Franke P, and Tsetlin V I (1997) Substance P derivatives with photoactivatable labels in the N-terminal part of the molecule. Journal of Peptide Research 50:408-414.

26. Smith R A, and Knowles J R (1973) Aryldiazirines. Potential reagents for photolabeling of biological receptor sites. Journal of the American Chemical Society 95:5072-5073.

27. Kojro E, Eich P, Gimpl G, and Fahrenholz F (1993) Direct identification of an extracellular agonist binding site in the renal V2 vasopressin receptor. Biochemistry 32:13537-13544.

28. Utkin Y N, Krivoshein A V, Davydov V L, Kasheverov I E, Franke P, Maslennikov I V, Arseniev A S, Hucho F, and Tsetlin V I (1998) Labeling of Torpedo californica nicotinic acetylcholine receptor subunits by cobratoxin derivatives with photoactivatable groups of different chemical nature at Lys23. European Journal of Biochemistry 253:229-235.

29. Wilson C J, Husain S S, Stimson E R, Dangott L J, Miller K W, and Maggio J E (1997) p-(4-Hydroxybenzoyl)phenylalanine: a photoreactive amino acid analog amenable to radioiodination for elucidation of peptide-protein interaction. Application to substance P receptor. Biochemistry 36:4542-4551.

30. Spiess, J., Rivier, J., Rivier, C. & Vale, W. (1981) Proc. Natl. Acad. Sci. USA 78,6517-6521.

31. Vale, W., Spiess, J., Rivier, C. & Rivier, J. (1981) Science 213, 1394-1397.

32. Eckart, K., Radulovic, J., Radulovic, M., Jahn, O., Blank, T., Stiedi, O. & Spiess, J. (1999) Curr. Med. Chem. 6, 1035-1053.

33. Perrin, M. H. & Vale, W. W. (1999) Annals of the New York Academy of Sciences 885, 312-328.

34. Potter, E., Behan, D. P., Fischer, W. H., Linton, E. A., Lowry, P. J. & Vale, W. W. (1991) Nature 349,423-426.

35. Potter, E., Behan, D. P., Linton, E. A., Lowry, P. J., Sawchenko, P. E. & Vale, W. W. (1992) Proc. Natl. Acad. Sci. USA 89, 4192-4196.

36. Seasholtz, A. F., Burrows, H. L., Karolyi, I. J. & Camper, S. A. (2001) Peptides 22, 743-751.

37. Behan, D. P., Heinrichs, S.C., Troncoso, J. C., Liu, X. J., Kawas, C. H., Ling, N. & De, S. E. B. (1995) Nature 378, 284-287.

38. Radulovic, J., Rühmann, A., Liepold, T. & Spiess, J. (1999) J. Neurosci. 19, 5016-5025.

39. Smith, G., Aubry, J.-M., Dellu, F., Contarino, A., Bilezikjian, L., Gold, L., Chen, R., Marchuk, Y., Hauser, C., Bentley, C., Sawchenko, P., Koob, G., Vale, W. & Lee, K.-F. (1998) Neuron 20,1093-1102.

40. Timpl, P., Spanagel, R., Sillaber, I., Kresse, A., Reul, J., Stalla, G. K., Blanquet, V., Steckler, T., Holsboer, F. & Wurst, W. (1998) Nat. Genet. 19, 162-166.

41. Kishimoto, T., Radulovic, J., Radulovic, M., Lin, C. R., Schrick, C., Hooshmand, F., Hermanson, O., Rosenfeld, M. G. & Spiess, J. (2000) Nat. Genet. 24,415-419.

42. Bale, T. L., Contarino, A., Smith, G. W., Chan, R., Gold, L. H., Sawchenko, P. E., Koob, G. F., Vale, W. W. & Lee, K.-F. (2000) Nat. Genet. 24, 410-414.

43. Sutton, S. W., Behan, D. P., Lahrichi, S. L., Kaiser, R., Corrigan, A., Lowry, P., Potter, E., Perrin, M. H., Rivier, J. & Vale, W. W. (1995) Endocrinology 136, 1097-1102.

44. Woods, R. J., Kennedy, K. M., Gibbins, J. M., Behan, D., Vale, W. W. & Lowry, P. J. (1994) Endocrinology 135, 768-73.

45. Jahn, O., Eckart, K., Sydow, S., Hofmann, B. A. & Spiess, J. (2001) Peptides 22, 47-56.

46. Eckart, K., Jahn, O., Radulovic, J., Tezval, H., van Werven, L. & Spiess, J. (2001) Proc. Natl. Acad. Sci. USA 98,11142-11147.

47. Tuinman, A. A. & Pettit, G. R. (1990) Int J. Peptide Protein Res. 36, 331-334.

48. Cuff, J. A. & Barton, G. J. (1999) Proteins 34, 508-519.

49. Henriot, S., Dautzenberg, F. M. & Kilpatrick, G. J. (1999) Eur. J. Pharmacol. 376, 321-324.

50. Dormán, G. & Prestwich, G., D. (1994) Biochemistry 33, 5661-5673.

51. Jahn, O., Hofmann, B., Brauns, O., Spiess, J. & Eckart, K. (2002) Int. J. Mass Spectrom. 214, 37-51.

52. Linton, E. A., Perkins, A. V., Woods, R. J., Eben, F., Wolfe, C. D., Behan, D. P., Potter, E., Vale, W. W. & Lowry, P. J. (1993) J. Clin. Endocrinol. Metabol. 76, 260-2.

53. Behan, D. P., Khongsaly, O., Liu, X. J., Ling, N., Goland, R., Nasman, B., Olsson, T. & Desouza, E. B. (1996) J. Clin. Endocrinol. Metabol. 81, 2579-2586.

54. Papayannopoulos, I. A. (1995) Mass Spectrom. Reviews 14, 49-73.

55. O'Neil, K. T., Erickson-Viitanen, S. & DeGrado, W. F. (1989) J. Biol. Chem. 264,14571-8.

56. Behar, V., Bisello, A., Bitan, G., Rosenblatt, M. & Chorev, M. (2000) J. Biol. Chem. 275, 9-17.

57. Li, H., Macdonald, D. M., Hronowski, X., Costello, C. E., Leeman, S. E. & Boyd, N. D. (2001) J. Biol. Chem. 276,10589-93.

58. Pallai, P. V., Mabilia, M., Goodman, M., Vale, W. & Rivier, J. (1983) Proc. Natl. Acad. Sci. USA 80, 6770-6774.

59. Romier, C., Bernassau, J., Cambillau, C. & Darbon, H. (1993) Protein Eng. 6,149-156.

60. Valverde, R. A., Seasholtz, A. F., Cortright, D. N. & Denver, R. J. (2001) Mol. Cell. Endocrinol. 173, 2940.

1. A ligand of the Corticotropin-Releasing Factor (CRF)-binding protein (CRFBP) selected from the group consisting of CRF, Urocortin (Ucn), [Ala²¹]Svg and Urotensin I, said ligand comprising a covalently linked benzophenone moiety.

2. A functional fragment of the ligand of claim 1 comprising a covalently linked benzophenone moiety.

3. The functional fragment of claim 2 selected from the group consisting of [Ala²¹]Svg⁵⁻³³, Urotensin I⁶⁻³³, CRF⁶⁻³³ and/or Ucn⁵⁻³².

4. The ligand of any one of claims 1 to 3, wherein said benzophenone moiety is covalently linked to the α-amino group of said ligand.

5. The ligand of claim 4, wherein said benzophenone moiety is covalently linked to the α-amino group of said ligand via an N-hydroxysuccinimide ester of said benzophenone moiety.

6. The ligand of claim 5, wherein said benzophenone moiety is provided by para-benzoylbenzoic acid or para-hydroxybenzoylbenzoic acid.

7. The ligand of any one of claims 1 to 3, wherein the histidine residue at position 32 of said CRF⁶⁻³³ is replaced by L-para-benzoylphenylalanine.

8. The ligand of claim 7, wherein para-benzoylbenzoic acid or para hydroxybenzoylbenzoic acid is covalently linked to the α-amino group of said ligand.

9. The ligand of claim 1 further comprising at the N-terminus a tyrosine residue to which said benzophenone moiety is covalently linked.

10. The ligand of claim 1 which is labeled.

11. The ligand of claim 9, wherein said tyrosine residue is labeled.

12. The ligand of claim 11, wherein said tyrosine residue is radioactively labeled.

13. The ligand of claim 12, wherein said tyrosine residue is labeled with ¹²⁵I.

14. The ligand of claim 1, wherein said CRFBP is rat CRFBP (rCRFBP), human CRFBP (hCRFBP), murine CRFBP (mCRFBP), sheep CRFBP or CRFBP of Xenopus laevis.

15. The ligand of claim 1, wherein said CRF is human/rat CRF (h/rCRF), murine CRF, porcine CRF, bovine CRF Tilipia CRF, frog CRF, sucker CRF, sockey salmon CRF or sockey salmon CRF and/or said CRF⁶⁻³³ is human/ratCRF⁶⁻³³ (h/r CRF⁶⁻³³), murineCRF⁶⁻³³, porcine CRF⁶⁻³³, bovineCRF⁶⁻³³ Tilipia CRF⁶⁻³³, frog CRF⁶⁻³³, sucker CRF⁶⁻³³ or sockey salmon CRF⁶⁻³³).

16. The ligand of claim 1, wherein said Ucn is ratUcn (rUcn), human Ucn, murine Ucn, ovine Ucn or hamster Ucn.

17. The ligand of claim 1 which is fused to another moiety.

18. A process for the purification of a CRFBP which comprises reacting said CRFBP with the ligand of claim 1, performing photoaffinity labeling, and purifying the resultant photoreaction products by HPLC.

19. A process for the characterization of the binding site of a CRFBP which comprises purifying the CRFBP according to the process of claim 18, fragmenting the purified product and determining the amino acid sequence of the relevant fragment.

20. Use of the ligand of claim 1 for detecting CRFBP.

21. The use of claim 20, wherein the detection is performed in the presence of CRF receptors.

22. The use of claim 20 or 21 wherein CRFBP is detected in a biological fluid.

23. Use of the ligand of claim 1 for identifying the binding site in CRFBP of a CRF or aCRF-like peptide.

24. Kit, comprising at least one of the ligands of claim 1.

25. A method for identifying an inhibitor for the binding of the ligand of claim 1 to CRFBP comprising:
(a) contacting a ligand of claim 1 and a CRFBP with a compound or a plurality of compounds to be screened;
(b) irradiating the mixture described in (a) with UV light above 300 nm including a significant emission at about 360 nm for effective excitation of the benzophenon photophore; and
(c) determining whether the compound or said plurality of compounds effects an inhibitory effect on the binding of said ligand and said CRFBP.

26. The method of claim 25, further comprising the step of:
(d) determining whether said compound or said plurality of compounds is able to bind to CRFR1 and/or CRFR2; wherein no or low binding to CRFR1 and/or CRFR2 indicates an inhibitor.
